(19) United States
(12) Patent Application Publication
(10) Pub. No.: US 2011/0130706 A1
(43) Pub. Date: Jun. 2, 2011

(54) SEQUENTIAL BODY SURFACE TREATMENT

(75) Inventors: Scott C. KELLOGG, Mattapoisett, MA (US); Shikha P. BARMAN, Bedford, MA (US); Seth M. LEDERMAN, New York, NY (US)

(73) Assignee: FOLLICA, INC., WALTHAM, MA (US)

(21) Appl. No.: 12/950,087

(22) Filed: Nov. 19, 2010

Related U.S. Application Data
(60) Provisional application No. 61/262,820, filed on Nov. 19, 2009.

(51) Int. Cl. A61M 37/00

(52) U.S. Cl. 604/20; 604/22

(57) ABSTRACT

The present methods and systems provide treatment of a preselected body surface by injuring a portion of the body surface, optionally applying a composition such as a pharmaceutical agent to the injured portion of the body surface, and selection of a further portion on the body surface for the same or different injury and optional contacting with the same or different composition, wherein the further portion has a preselected geometry with respect to the first injured portion. The repetition of the selection, injury, and optional exposure to a composition with respect to successive portions of the body surface, optionally under partial or total computer control, provides efficient treatment of the body surface pursuant to the objective of regeneration, remodeling, resurfacing, restoration, follicular neogenesis, neocollagenesis, stem cell recruitment, activation, or differentiation, reepitheliazation, wound healing, or any other desired biological or physical modification.

### CROSS-REFERENCE TO RELATED APPLICATION

The present application claims priority to U.S. Provisional App. No. 61/262,820, filed Nov. 19, 2009, the entire contents of which are incorporated herein by reference.

### TECHNICAL FIELD

The present invention pertains to the injury to a body surface pursuant to the induction of follicular neogenesis or another cosmetic or medical treatment.

### BACKGROUND

Follicular neogenesis is the generation of new hair follicles after birth. Human beings are born with a full complement of hair follicles, which can change in size and growth characteristics (as in early baldness) or can ultimately degenerate and disappear (as in the late stages of baldness or in permanent scarring or cicatricial alopecias). The generation of new hair follicles is desirable in the treatment of common baldness as well as less common conditions that are characterized by hair loss, such as discoid lupus, erythematosis, congenital hypotrichosis, lichen planopilaris, and other scarring alopecias, among other conditions. New follicles are either from new cells or from divisions of existing follicles.

The reduction or elimination of unwanted hair is also of widespread interest, most prominently among women but increasingly among men as well. Hypertrichosis, excess hair in androgen-dependent areas of the skin, idiopathic hirsutism, female post-menopausal facial hair, axillary hair, leg hair, back hair, ear hair, nose hair, and other conditions may give rise to the desire for hair removal treatment. Current methods for the reduction or elimination of hair may involve depilation and epilation with or without the use of hair growth retardants. Electrology (electrolysis), laser and intense pulsed light are also used for permanent hair removal. However, multiple sessions with trained medical personnel are typically required.

Techniques such as microdermabrasion and laser treatment have been used to reduce or eliminate the appearance of various cosmetically undesirable skin conditions, such as wrinkling and other aging-related features, scarring, moles, birthmarks, and assorted types of abnormal skin pigmentation.

Although such treatments may yield successful results when respectively used on an individual basis, there has been little, if any progress in developing systems that integrate multiple treatment modalities in order to provide more efficient and comprehensive therapeutic regimes.

### SUMMARY

The present disclosure provides methods and systems that permit treatment of body surface that is particularized with respect to the type of body surface, the features present at the body surface, and the individual needs of the subject. The use of iterative selection and treatment of respective target areas on a body surface overcomes the limitations of prior techniques that did not employ methodical and precise treatment of discrete areas or the choice from among different treatment types during respective iterations. By treating discrete areas of a body surface by correspondingly discrete means and optionally combining multiple treatment modalities within a single protocol/system, the present systems and methods enhance the efficiency of the treatment of a body surface for any of a number of different therapeutic ends, including regeneration, remodeling, resurfacing, restoration, follicular neogenesis, neocollagenesis, stem cell recruitment, activation, or differentiation, reepitheliazation, wound healing, or any other desired biological or physical modification.

In one aspect, methods are provided for treating a preselected body surface comprising injuring a first target area on the body surface; optionally applying a composition to the injured first target area; selecting a further target area on the body surface having a preselected geometry with respect to the first target area; injuring the further target area on the body surface; and optionally applying the same or a different composition to the injured further target area.

In another aspect, systems for treating a body surface are provided comprising a traumatizer for inducing injury to a first target area at the body surface; an applicator for delivering a composition to the first target area; wherein the traumatizer, the applicator, or both are under the operative control of a general purpose digital computer; and wherein the computer is configured for selecting a further target area on the body surface having a preselected geometry with respect to the first target area.

### BRIEF DESCRIPTION OF THE DRAWINGS

FIG. 1 illustrates the use of a fractional laser to form a hole in human skin, after which the hole is filled with a highly viscous drug-containing gel via an ink-jet precision fill device; body heat or other external factors then crosslink the gel into a stable drug-releasing matrix.

FIG. 2 shows a component of the present invention that features an integrated head design.

### DETAILED DESCRIPTION OF ILLUSTRATIVE EMBODIMENTS

The present inventions may be understood more readily by reference to the following detailed description taken in connection with the accompanying figures and examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

It is intended that any component, element, attribute, or step that is positively recited herein may be explicitly excluded in the claims, whether such components, elements, attributes, or steps are listed as alternatives or whether they are recited in isolation.

In the present disclosure the singular forms “a,” “an,” and “the” include the plural reference, and reference to a particular numerical value includes at least that particular value, unless the context clearly indicates otherwise. Thus, for example, a reference to “a composition” is a reference to one or more of such compositions and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

The disclosures of each patent, patent application, and publication cited or described in this document are hereby incorporated herein by reference, in their entirety.

The wounding of skin by physical means such as microdermabrasion, dermabrasion, and varying degrees of tissue disruption or excision can create a biological milieu of stem cells and inflammatory factors and signaling molecules, the interplay of which can result in neocollagenesis and neofollicles. Deliberate wounding of skin or other body surfaces can also be used to effect changes in surface appearance and repairs of defects through regeneration of lost or deficient tissue components. The present disclosure envisions the creation of the optimum biological conditions by induction of one or more preselected types of injury to a target area of tissue in an iterative manner in order to provide a beneficial treatment regime with respect to a desired body surface. The present systems and methods are distinguishable from prior methodologies pursuant to which multiple treatment sessions are necessitated because of the inability of any single system to provide multiple treatment modalities. These and other advantages will become apparent throughout the present disclosure.

In one aspect, methods are provided for treating a preselected body surface comprising injuring a first target area on the body surface; optionally applying a composition to the injured first target area; selecting a further target area on the body surface having a preselected geometry with respect to the first target area; injuring the further target area on the body surface; and optionally applying the same or a different composition to the injured further target area.

The body surface may be an exterior or an interior surface. Skin surfaces of all types, for example, facial skin, the scalp, or skin on the back, legs, or arms, may be subjected to treatment in accordance with the present disclosure. “Interior” surfaces (i.e., those that are substantially within the body) may include those in the oral cavity (such as the palate, the buccal surfaces, or the gums), trachea, pharynx, esophagus, stomach, small or large intestine, the surface of any organ, a blood vessel, or any other interior surface that is directly or indirectly accessible to one or each of the components necessary for injuring and optionally applying a composition, as described more fully herein.

Thus, in accordance with certain embodiments of the present invention, therapeutic, cosmetic or other action is accomplished upon the surface of an organ, system, or organelle of a subject. Such surface may be effectively external to the subject, such as the skin, ear canal, nare, or eye. The surface may also be internal to the subject. Thus, such action may be effected upon a surface which may be reached by simple probing. Examples include surfaces of the rectum, colon, throat, esophagus, stomach, trachea, or bronchus. Additionally, access to a surface for action may employ arterial access, laparoscopic access, or traditional surgical access. In this way, a very large number of surfaces internal to a subject may be treated.

Next, a first target area on the body surface is injured. The target area may be some part or the entirety of a physical feature of which treatment is desired.

The physical feature may be one that is typically present at the type of body surface of which treatment is desired. For example, if the body surface is the scalp, the physical feature may be a hair, a vellus hair, a hair pore, a sweat gland, an area of pigmentation, scar tissue, a wound, a “featureless” patch of skin (i.e., an area of skin without any of the preceding features), or another normal or abnormal physical feature that is known to occur at the scalp. Likewise, if the body surface is facial skin, the physical feature may be a hair, a miniaturized hair, a vellus hair, a hair pore, a sweat gland, an area of pigmentation, scar tissue, a wound, a blood vessel, a wrinkle, a wart, a “featureless” patch of skin (i.e., an area of skin without any of the preceding features), or another normal or abnormal physical feature that is known to occur on facial skin. If the body surface is the interior surface of the esophagus, the physical feature may be a lesion, a node, diverticula, blood vessels, a “featureless” area of tissue (i.e., an area of tissue without any of the preceding features), or another normal or abnormal physical feature that is known to occur at the interior surface of the esophagus.

Other physical features my include aging-related skin conditions, pigmentation disorders, acne, stretch marks, skin disorders (such as psoriasis, leprosy, atopic dermatitis, or other conditions resulting from an autoimmune disorder), skin infections, skin lesions, keloids.

“Aging-related skin condition” may refer to a condition resulting from intrinsic aging (i.e., chronological aging) as well as extrinsic aging (i.e., resulting from environmental conditions such as photoaging). Examples of such conditions are wrinkles (e.g., fine and coarse wrinkles), brown spots, dyspigmentation, laxity, yellow hue, telangiectasia, leathery appearance, and cutaneous malignancies. Wrinkles and skin laxity are primarily caused by a decrease in the subcutaneous fat layer combined with decreased collagen and elastin synthesis in the dermis. Alterations in skin pigmentation (e.g., brown spots and dyspigmentation) are related to altered melanocyte function and changes in melanin accumulation within basal keratinocytes. Changes in skin blood vessel dilation and distribution contribute to the appearance of telangiectasia and spider veins. Increased skin malignancies are also associated with increased skin aging and generally result from a combination of environmental exposure (i.e., high UV exposure prior to age 18) and genetics. A reduction of sweat gland number and function is another age-related skin condition.

“Pigmentation disorder” refers to a skin or hair condition arising from abnormal skin or hair pigmentation that may but need not be caused by alterations in melanocyte function or viability. Such disorders include abnormal pigmentation in humans such as albinism, melasma, vitiligo, hair graying, freckles, hemochromatosis, hemosideriosis, and tinea versicolor.

“Acne” generally refers to a skin condition arising from the pilosebaceous unit characterized by hyperkeratinization, P. acnes infection, and abnormal sebum production and that results in a visible skin lesion.

The injuring of the first target area may be via any modality that is suitable for inducing regeneration, remodeling, resurfacing, restoration, follicular neogenesis, neocollagenesis, stem cell recruitment, activation, or differentiation, reepitheliazation, wound healing, or any other desired biological or physical modification. The injury may be induced by any mechanical, chemical, energetic, sound- or ultrasound-based, or electromagnetic means. Injury may achieved through abrasion (e.g., by rubbing or wearing away), perforation, burning, stripping, or by any method that results in disturbing the intactness of the body surface.

The injury may comprise the removal of a column, slice, wedge, cube, plug, or other portion of tissue at the target area to form a “channel.” The channel may extend from the body surface to a depth of about 0.5 mm to about 4 mm below the surface, or to any other desired depth, wherein the channel may be oriented perpendicular or at an oblique angle relative to the body surface. The removal of a column of tissue at the target area may be accomplished by any suitable technique, including a fractional ablative laser, a punch biopsy needle, a microneedle, a micro-coring needle, or another suitable modality. Removal of a column of tissue may invoke a full thickness skin excision (FTE) model to establish a skin healing state that is conducive to follicular neogenesis by removing all tissue components and relying on de novo hair follicle formation. The channels that are formed pursuant to this type of injury are surrounded by intact skin with viable keratinocytes and melanocytes. Due to the proximity of the viable cells to the site of injury, the re-epithelialization process is more rapid than bulk ablation of tissue over a large area. The standard FTE model is created with a scalpel in animal models. This aggressive procedure does not lend itself directly to commercialization due to risk of scarring. However, various fractional laser modalities may be used to achieve this deeper disruption on a grid pattern. A fractional laser may be use to “drill”, for example, 1 mm diameter holes with a 1 mm hole spacing. Although tissue is completely removed within the 1 mm hole, the surrounding intact tissue prevents scarring and therefore the FTE model is invoked within each hole.

A fractional like hole pattern can also be achieved with an array of punch biopsy needles. For example, 1 mm punch biopsies can be arranged with 1 mm hole spacing. When inserted into the scalp, the cored skin samples can be removed and as in above, the FTE model is invoked within each hole. Similarly, and for smaller holes, micro needles and micro-coring needles could be used. Micro-roller needle devices already on the market, may be used to create the fractional injury pattern. Other modalities such as ultrasound, electroporation, RF ablation, and electromagnetic fields can all be used to perturb and/or remove the tissue of a body surface such that the aforementioned models are invoked.

Other injury types may invoke a microdermabrasion model that induces reorganization of existing body surface components. Where the body surface is skin, such components may include follicular structures. As used herein “dermabrasion” and “microdermabrasion”, and “integumental disruption” refer to the techniques and devices that are associated with such terminology in accordance with the skill of routineers in the art, and not necessarily the application of such techniques and devices to the skin. Thus, the inclusion of the roots “derm” and “integument” should not be construed as limiting the use of applicable techniques and devices to the skin, as such use may be in connection with the injury of any body surface. The microdermabrasion model is substantially superficial and may have a clinical endpoint that is characterized by pinpoint bleeding. Where the body surface is skin, the microdermabrasion model may include removal of the stratum corneum and epidermis. Standard dermabrasion, for example, by use of an abrasive wheel or an abrasive cloth, may be used to achieve the desired clinical endpoint in this injury model. Lasers may be used to invoke this model as well. Standard CO₂ or YAG/Erbium lasers may be used for this purpose by selecting the appropriate depth of body surface disruption; for skin, this involves the removal of the stratum corneum and epidermis. Other techniques for dermabrasion and integumental perturbation are described infra. This type of injury may be selected in order to induce a state that is conducive to follicular neogenesis, neocollagenesis, or both, for cosmetic skin resurfacing, or for another cosmetic or restorative purpose.

While the popularity of mechanical dermabrasion has decreased in recent years with the advent of laser-based procedures, dermabrasion is still used for removing features on the skin such as facial scars resulting from acne and other trauma. Small, portable mechanical dermabrasion equipment uses interchangeable diamond fraises operated at different rotation speeds, for example, to remove the epidermis and dermis to differing skin depths levels. Adult human skin treated with dermabrasion completely re-epithelializes in 5-7 days with minor redness lasting up to a few weeks. Dermabrasion may be carried out using any technique known in the art. For example, dermabrasion may be carried out using an abrasive wheel to, in some embodiments, achieve pinpoint bleeding. In other embodiments, dermabrasion may be carried out using an abrasive wheel to achieve larger globules of bleeding and frayed collagen. In some embodiments, dermabrasion is accomplished by removal of surface skin by particle bombardment, for example, with alumina-, ice- or silica-based particles, or even particles comprising a pharmaceutically active ingredient, such as lithium (as discussed more fully infra). For example, micron-sized particles are propelled toward the surface of the skin via short strokes of a handpiece, such as a particle gun, as known in the art. The velocity of particles is controlled through positive or negative pressure. The depth of body surface, e.g., skin, removed by the procedure is a function of the volume of particles impacting the body surface, the suction or positive pressure, the speed of movement of the handpiece, and the number of passes per area of the body surface. Non-powered devices such as abrasive cloths can also be used to achieve the dermabrasion, with the optional achievement of the same endpoint. Other means for dermabrasion and integumental perturbation are discussed below.

In some embodiments, dermabrasion is achieved by using a device for microdermabrasion to the point where treatment is stopped upon the observation of pinpoint bleeding; in skin, this endpoint signals the removal of the stratum corneum and epidermis into the papillary dermis. In other embodiments, dermabrasion is achieved by using a device for microdermabrasion to the point where treatment is stopped upon the observation of larger globules of bleeding and frayed collagen, which, in skin, signals the removal of the stratum corneum and epidermis into the papillary and reticular dermis. In some embodiments, this extended use is reduced by using a microdermabrasion device with increased output pressure and increased abrasion particle size, which may accelerate the tissue removal/perturbation process.

Where the body surface is skin, integumental perturbation by one or more of the aforementioned methods achieves removal of part or all of the epidermis. In some embodiments, integumental perturbation removes the entire epidermis. In some embodiments, integumental perturbation removes the papillary dermis. In some embodiments, integumental perturbation removes the reticular dermis. The depth of integumental perturbation depends on the thickness of the skin at a particular treatment area. For example, the skin of the eyelid is significantly thinner than that of the scalp. The occurrence of pinpoint bleeding indicates that the epidermis and portions of the dermis have been removed. Deeper penetration can results in much more bleeding, and the perturbation can go as deeps as the hypodermis.

In some embodiments, perturbation by one or more of the aforementioned methods is to a body surface depth of 60 μm. In some embodiments, perturbation is to a body surface depth of 60-100 μm. In some embodiments, perturbation is to a body surface depth of 100 μm. In some embodiments, perturbation is to a body surface depth of 100-500 μm. In some embodiments, perturbation is to a body surface depth of 100 μm. In some embodiments, perturbation is to a body surface depth of 1 mm or more. In some embodiments, perturbation is to a body surface depth of 1 mm to 3 mm. In some embodiments, perturbation is to a body surface depth of 1 mm to 5 mm.

As provided above, integumental perturbation can be achieved by any means known in the art or described herein, such as, for example, using chemical or mechanical means. In one embodiment, integumental perturbation comprises disrupting the skin of the subject (for example, resulting in the induction of re-epithelialization of the skin of the subject). In some embodiments, when the body surface is skin, a certain area of the epithelium is partially or wholly disrupted. In some embodiments, a certain area of both the epithelium and stratum corneum are partially or wholly disrupted. For a discussion of skin disruption and re-epithelialization, including methods for disrupting skin and inducing and detecting re-epithelialization, see PCT Publication Nos. WO 2008/042216 and WO 2006/105109, each of which is incorporated herein by reference. Integumental perturbation can be used to induce, for example, a burn, excision, dermabrasion, full-thickness excision, or other form of abrasion or wound.

Mechanical means of integumental perturbation include, for example, use of sandpaper, a felt wheel, ultrasound, supersonically accelerated mixture of saline and oxygen, tape-stripping, spiky patch, or peels. Chemical means of integumental perturbation can be achieved, for example, using phenol, trichloroacetic acid, or ascorbic acid. Electromagnetic means of integumental perturbation include, for example, use of a laser (e.g., using lasers, such as those that deliver ablative, non-ablative, fractional, non-fractional, superficial or deep treatment, and/or are CO₂-based, or Erbium-YAG-based, etc.). Integumental perturbation can also be achieved through, for example, the use of visible, infrared, ultraviolet, radio, or X-ray irradiation. Electrical or magnetic means of disruption of the body surface can be achieved, for example, through the application of an electrical current, or through electroporation or RF ablation. Electric or magnetic means can also include the induction of an electric or a magnetic field, or an electromagnetic field. For example, an electrical current can be induced in the skin by application of an alternating magnetic field. A radiofrequency power source can be coupled to a conducting element, and the currents that are induced will heat the skin, resulting in an alteration or disruption of the skin. Integumental perturbation can also be achieved through surgery, for example, a biopsy, a skin transplant, hair transplant, cosmetic surgery, etc.

In some embodiments, integumental perturbation is by laser treatment, as discussed in below. In a preferred embodiment, integumental perturbation by laser treatment is by a fractional laser, using, e.g., an Erbium-YAG laser at around 1540 nm or around 1550 nm (for example, using a Fraxel® laser (Solta Medical)). Treatment with an Erbium-YAG laser at 1540 or 1550 nm is typically non-ablative, and pinpoint bleeding typical of laser treatment is not observed since the outer portion of the body surface (for example, in skin, the stratum corneum) is left intact. The column of dead cells (for skin, epidermal and/or dermal) in the path of the laser treatment is termed a “coagulum.” In another embodiment, integumental perturbation by laser treatment is by a fractional laser, using, e.g., a CO₂ laser at 10,600 nm. Treatment with a CO₂ laser at 10,600 nm is typically ablative, and typically leads to the appearance of pinpoint bleeding.

A standard CO₂ or Erbium-YAG laser can be used to create superficial and, optionally, broad based, integumental perturbation similar to dermabrasion (discussed below). Although the pinpoint bleeding clinical endpoint may not be achieved due to the coagulation properties of (particularly non-ablative) lasers, use of a laser has an advantage making it possible to select the specific depth of body surface disruption to effectively remove the outer portions (e.g., stratum corneum) and internal portions (e.g., epidermis), or parts thereof.

In one embodiment, the laser treatment is ablative. For example, full ablation of tissue is generated by the targeting of tissue water at wavelengths of 10,600 nm by a CO₂ laser or 2940 nm by an Erbium-YAG laser. With respect to skin, in this mode of laser treatment the epidermis is removed entirely and the dermis receives thermal tissue damage. The depth of tissue ablation may be a full ablation of the epidermis, or a partial ablation of the epidermis, with both modes causing adequate wounding to the skin to induce the inflammatory cascade requisite for regeneration. In another variation, the depth of ablation may extend partially into the dermis, to generate a deep wound. The denuded skin surface is then treated with a composition described herein; alternatively, the composition can be delivered into the skin after the initial re-epithelialization has occurred already, to prevent clearance and extrusion of any drug-containing depots from the tissue site by the biological debris-clearance process. In one embodiment, a composition described herein is delivered by a sustained release depot that is comprised of biocompatible, bioabsorbable polymers that are compatible to tissue.

As disclosed supra, an full thickness excision model may be invoked by use of a fractional laser.

In some embodiments, the laser treatment is ablative and fractional. For example, fractional tissue ablation can be achieved using a CO₂ laser at 10,600 nm or an Erbium-YAG laser at 2940 nm (e.g., the Lux 2940 laser, Pixel laser, or Profractional laser). In some such embodiments, the lasing beam creates micro-columns of thermal injury into the body surface, at depths up to 4 mm and vaporizes the tissue in the process. Ablative treatment with a fractional laser leads to ablation of a fraction of the body surface leaving intervening regions of normal tissue intact, which in skin allows for rapid repopulation of the epidermis. Approximately 15%-25% of the body surface is treated per session. The density of micro thermal zones (MTZ) can be varied to create a dense “grid” of injury columns surrounded by intact tissue and viable cells. The density of the grid on the treatment area plays an important role. The denser the grid, the more the thermal injury and the type of injury begins to approximate full ablation. Therefore, it is appreciated that there may be an “optimum” MTZ density that is appropriate for use in the methods disclosed herein. In one embodiment, a composition described herein is delivered into the dermis immediately after wounding, or after initial re-epithelialization has occurred.

In another embodiment, the mode of laser treatment is non-ablative, wherein outer portions of the body surface (e.g., in skin, the stratum corneum and the epidermis) are intact after treatment, with subsurface portions (e.g., dermis) selected for the deep thermal treatment required for the requisite injury to tissue. This can be accomplished by cooling the epidermis during the laser treatment. For example, one could use the timed cooling of the outer portions of the body surface with a cryogen spray while the laser delivers deep thermal damage to the subsurface portions. In this application, the depth of treatment may be 1 mm to 3 mm into the body surface. One could also use contact cooling, such as a copper or sapphire tip. Lasers that are non-ablative have emission wavelengths between 1000-1600 nm, with energy fluences that will cause thermal injury, but do not vaporize the tissue. The non-ablative lasers can be bulk, wherein a single spot beam can be used to treat a homogenous section of tissue. In some embodiments, multiple treatments are required to achieve the desired effect. In one embodiment, a composition (e.g., a lithium composition) described herein is delivered deep into the dermis in polymeric micro-depots and released in a sustained fashion. Lasers that are non-ablative include the pulsed dye laser (vascular), the 1064 Nd:YAG laser, or the Erbium-YAG laser at 1540 nm or 1550 nm (e.g., the Fraxel® laser). Use of an Erbium-YAG laser at around 1540 nm or around 1550 nm, as opposed to its use at 2940 nm, “coagulates” zones of dermis and epidermis (forming a “coagulum”) and leaves the stratum corneum essentially intact.

In another embodiment, the mode of laser treatment is fractional and non-ablative. Treatment with a fractional, non-ablative laser leads to perturbation of a fraction of the body surface, leaving intervening regions of normal tissue intact (which in skin, allows for rapid repopulation of the epidermis). Approximately 15%-25% of the body surface is treated per session. As in any non-ablative process, the barrier function is maintained, while deep thermal heating of subsurface portions can occur. For example, in skin, zones of dermis and epidermis are coagulated and the stratum corneum is left essentially intact. This process has been coined “fractional photothermolysis” and can be accomplished, e.g., using the Erbium-YAG laser with an emission at or around 1540 nm or 1550 nm. In one embodiment, a composition described herein (e.g., a lithium composition) is delivered immediately after the tissue injury, deep into the body surface (in skin, into the dermis). In another embodiment, a combination of bulk and fractional ablation modes of tissue injury are used.

Another injury type may involve the segmentation of a hair follicle into at least two disunited subunits. The injury that is used for segmentation of a hair follicle into disunited subunits may include the application of an incisor at an oblique angle relative to the body surface to a depth below the body surface that is sufficient to intersect and cross the follicle. In some embodiments, incisor is applied at an angle of 89°, 85°, about 80°, about 75°, about 70°, about 65°, about 60°, about 55°, about 50°, about 45°, about 40°, about 35°, about 30°, about 25°, about 20°, about 15°, about 10°, about 5°, or less relative to the body surface. The incisor may be applied at an angle φ relative to axis y that is perpendicular to the body surface, wherein the hair follicle is oriented at an angle α relative to the body surface, wherein the sum of angle α and an angle β is 90°, and wherein the sum of angle φ and an angle β is about 65° to about 115°. In some instances, the sum of angle φ and angle β may be about 70°, about 75°, about 80°, about 85°, about 90°, about 95°, about 100°, about 105°, or about 110°.

The segmentation of a hair follicle by applying an incisor at an oblique angle relative to the body surface may alternatively comprise splicing a hair follicle substantially along its long axis. For example, given a hair follicle that is oriented at about 40° relative to the body surface, the incisor may be directed at a comparable angle against the body surface at the location of the follicle and parallel to the long axis of the follicle. The application of an incisor in this manner preferably functions to splice the follicle along its long axis into at least two portions (if two portions are produced, halves). Each portion of the spliced follicle contains all of the biological follicular components that are necessary to generate a complete follicle and produce hair. Thus, the splicing of a hair follicle in this manner can generate a pair of hair-producing follicles from a single follicle.

Optionally, further to the process of segmenting a hair follicle by applying an incisor at an oblique angle relative to the body surface, an incisor may also be applied substantially “downwards”, i.e., at about 90°, relative to the body surface in order to segment a further hair follicle that is oriented at a substantially similar angle relative to the body surface. The application of an incisor substantially downwards onto a hair follicle having this orientation preferably functions to splice the follicle into at least two substantially vertically oriented halves. Each half of the spliced follicle contains all of the biological follicular components that are necessary to generate a complete follicle and produce hair. Thus, the splicing of a hair follicle in this manner can generate a pair of hair-producing follicles from a single follicle.

The incisor may be any physical instrument, material, or form of energy that segments the follicle into at least two disunited subunits. For example, the incisor may be an ablative laser, a punch biopsy, a microneedle, or a micro-coring needle that results in the removal of a column of tissue to form a channel that transects the follicle. The incisor may also be a non-ablative laser that leaves a coagulum along its path but likewise transects and segments the follicle. In other embodiments, the incisor may be a high-pressure jet of fluid, such as water or gas, that penetrates the body surface and segments the follicle.

A composition may be applied to the injured first target area. Because the composition may be applied to the target area after or contemporaneously with the injuring of the first target area, the “injured first target area” refers to the target area as it is being subjected to injury or after it has been subjected to injury. As used herein, “contemporaneously” means that during at least part of the time that the first target area is being injured, the composition is applied to the first target area. Thus, if the injury is induced during a time period having a total duration of one second, applying a composition to the target area for 0.5 seconds after the target area is subjected to injury and for 0.1 seconds during the injury period will be considered to have been contemporaneous with the injuring of the target area.

The composition may comprise one or more physiologically active compounds. For example, the composition may include one or more of compounds that can influence the generation of hair follicles or the stimulation of hair growth, antioxidants, antihistamines, anti-inflammatory agents, anti-cancer agents, retinoids, anti-androgen agents, immunosuppressants, channel openers, antimicrobials, herbs, extracts, vitamins, co-factors, psoralen, anthralin, and antibiotics. The type of composition that is applied to the injured target area, the manner of application, or both may be selected from a set of compositions and methods of application that are appropriate for use with the type of injury to which the target area was subjected. For example, if the target area was injured in a manner that is intended to invoke a microdermabrasion model in order to induce follicular neogenesis, then the composition and mode of delivery may be tailored to this model and desired outcome.

Any compound or composition that can release a lithium ion is suitable for use in the present methods and systems. Such compounds include but are not limited to a pharmaceutically acceptable prodrug, salt or solvate (e.g., a hydrate) of lithium (sometimes referred to herein as “lithium compounds”). Optionally, the lithium compounds can be formulated with a pharmaceutically acceptable vehicle, carrier, diluent, or excipient, or a mixture thereof. Additionally, lithium-polymer complexes can be utilized to developed various sustained release lithium matrices.

Any form of lithium approved for pharmacological use may be used. For example, lithium is best known as a mood stabilizing drug, primarily in the treatment of bipolar disorder, for which lithium carbonate (Li₂CO₃), sold under several trade names, is the most commonly used. Other commonly used lithium salts include lithium citrate (Li₃C₆H₅O₇), lithium sulfate (Li₂SO₄), lithium aspartate, and lithium orotate. A lithium formulation well-suited for use in the composition is lithium gluconate, for example, a topical ointment of 8% lithium gluconate (Lithioderm™), is approved for the treatment of seborrhoeic dermatitis. See, e.g., Dreno and Moyse, 2002, Eur J Dermatol 12:549-552; Dréno et al., 2007, Ann Dermatol Venereol 134:347-351 (abstract); and Ballanger et al., 2008, Arch Dermatol Res 300:215-223, each of which is incorporated by reference herein in its entirety. Another lithium formulation is lithium succinate, for example, an ointment comprising 8% lithium succinate, which is also used to treat seborrhoeic dermatitis. See, e.g., Langtry et al., 1996, Clinical and Experimental Dermatology 22:216-219; and Cuelenaere et al., 1992, Dermatology 184:194-197, each of which is incorporated by reference herein in its entirety. In one embodiment, the lithium formulation is an ointment comprising 8% lithium succinate and 0.05% zinc sulfate (marketed in the U.K. as Efalith). See, e.g., Efalith Multicenter Trial Group, 1992, J Am Acad Dermatol 26:452-457, which is incorporated by reference herein in its entirety. Examples of lithium succinate formulations and other lithium formulations for use in the intermittent lithium treatments or pulse lithium treatment described herein are also described in U.S. Pat. No. 5,594,031, issued Jan. 14, 1997, which is incorporated herein by reference in its entirety.

Any pharmaceutically acceptable lithium salt may be used. It will be understood by one of ordinary skill in the art that pharmaceutically acceptable lithium salts are preferred. See, e.g., Berge et al., J. Pharm. Sci. 1977, 66:1-19; Stahl & Wermuth, eds., 2002, Handbook of Pharmaceutical Salts, Properties, and Use, Zurich, Switzerland: Wiley-VCH and VHCA; Remington's Pharmaceutical Sciences, 1990, 18^{th} eds., Easton, Pa.: Mack Publishing; Remington: The Science and Practice of Pharmacy, 1995, 19^{th} eds., Easton, Pa.: Mack Publishing.

In some embodiments, the compositions comprise mixtures of one or more lithium salts. For example, a mixture of a fast-dissolving lithium salt can be mixed with a slow dissolving lithium salt proportionately to achieve the release profile. In certain embodiments, the lithium salts do not comprise lithium chloride.

In some embodiments, the lithium salt can be the salt form of anionic amino acids or poly(amino) acids. Examples of these are glutamic acid, aspartic acid, polyglutamic acid, polyaspartic acid.

By reciting lithium salts of the acids set forth above, it is not intended to mean only the lithium salts prepared directly from the specifically recited acids. In contrast, the present disclosure encompasses the lithium salts of the acids made by any method known to one of ordinary skill in the art, including but not limited to acid-base chemistry and cation-exchange chemistry.

In another embodiment, lithium salts of anionic drugs that positively affect hair growth, such as prostaglandins can be administered. In another embodiment, a large anion or multianionic polymer such as polyacrylic acid can be complexed with lithium, then complexed with a cationic compound, such as finasteride, to achieve a slow release formulation of both lithium ion and finasteride. Similarly, a lithium complex with a polyanion can be complexed further with the amines of minoxidil, at pHs greater than 5.

Lithium compounds for use herein may contain an acidic or basic moiety, which may also be provided as a pharmaceutically acceptable salt. See, Berge et al., J. Pharm. Sci. 1977, 66:1-19; Stahl & Wermuth, eds., 2002, Handbook of Pharmaceutical Salts, Properties, and Use Zurich, Switzerland: Wiley-VCH and VHCA.

In some embodiments, the lithium salts are organic lithium salts. Organic lithium salts for use in these embodiments include lithium 2,2-dichloroacetate, lithium salts of acylated amino acids (e.g., lithium N-acetylcysteinate or lithium N-stearoylcysteinate), a lithium salt of poly(lactic acid), a lithium salt of a polysaccharides or derivative thereof, lithium acetylsalicylate, lithium adipate, lithium hyaluronate and derivatives thereof, lithium polyacrylate and derivatives thereof, lithium chondroitin sulfate and derivatives thereof, lithium stearate, linoleic acid, lithium lenoleate, lithium oleate, lithium taurocholate, lithium cholate, lithium glycocholate, lithium deoxycholate, lithium alginate and derivatives thereof, lithium ascorbate, lithium L-aspartate, lithium benzenesulfonate, lithium benzoate, lithium 4-acetamidobenzoate, lithium (+)-camphorate, lithium camphorsulfonate, lithium (+)-(1S)-camphor-10-sulfonate, lithium caprate, lithium caproate, lithium caprylate, lithium cinnamate, lithium citrate, lithium cyclamate, lithium cyclohexanesulfamate, lithium dodecyl sulfate, lithium ethane-1,2-disulfonate, lithium ethanesulfonate, lithium 2-hydroxy-ethanesulfonate, lithium formate, lithium fumarate, lithium galactarate, lithium gentisate, lithium glucoheptonate, lithium D-gluconate, lithium D-glucuronate, lithium L-glutamate, lithium α-oxoglutarate, lithium glycolate, lithium hippurate, lithium (+)-L-lactate, lithium (±)-DL-lactate, lithium lactobionate, lithium laurate, lithium (−)-L-malate, lithium maleate, lithium malonate, lithium (±)-DL-mandelate, lithium methanesulfonate, lithium naphthalene-2-sulfonate, lithium naphthalene-1,5-disulfonate, lithium 1-hydroxy-2-naphthoate, lithium nicotinate, lithium oleate, lithium orotate, lithium oxalate, lithium palmitate, lithium pamoate, lithium L-pyroglutamate, lithium saccharate, lithium salicylate, lithium 4-amino-salicylate, sebacic acid, lithium stearate, lithium succinate, lithium tannate, lithium (+)-L-tartarate, lithium thiocyanate, lithium p-toluenesulfonate, lithium undecylenate, or lithium valerate. In some embodiments, the organic lithium salt for use in these embodiments is lithium (S)-2-alkylthio-2-phenylacetate or lithium (R)-2-alkylthio-2-phenylacetate (e.g., wherein the alkyl is C2-C22 straight chain alkyl, preferably C8-16). See, e.g., International Patent Application Publication No. WO 2009/019385, published Feb. 12, 2009, which is incorporated herein by reference in its entirety.

The organic lithium salts may comprise the lithium salts of acetic acid, 2,2-dichloroacetic acid, acetylsalicylic acid, acylated amino acids, adipic acid, hyaluronic acid and derivatives thereof, polyacrylic acid and derivatives thereof, chondroitin sulfate and derivatives thereof, poly(lactic acid-co-glycolic acid), poly(lactic acid), poly(glycolic acid), pegylated lactic acid, stearic acid, linoleic acid, oleic acid, taurocholic acid, cholic acid, glycocholic acid, deoxycholic acid, alginic acid and derivatives thereof, anionic derivatives of polysaccharides, poly(sebacic anhydride)s and derivatives thereof, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, oleic acid, orotic acid, oxalic acid, palmitic acid, pamoic acid, L-pyroglutamic acid, saccharic acid, salicylic acid, 4-amino-salicylic acid, sebacic acid, stearic acid, succinic acid, tannic acid, (+)-L-tartaric acid, thiocyanic acid, p-toluenesulfonic acid, undecylenic acid, or valeric acid. Other organic lithium salts for use in these embodiments is the lithium salt of (S)-2-alkylthio-2-phenylacetic acid or the lithium salt of (R)-2-alkylthio-2-phenylacetic acid (e.g., wherein the alkyl is C2-C22 straight chain alkyl, preferably C8-16). See, e.g., International Patent Application Publication No. WO 2009/019385, published Feb. 12, 2009, which is incorporated herein by reference in its entirety.

In some embodiments of the present compositions, the organic lithium salt can be modified to create sustained release lithium salts. Due to the size of the lithium ion, it is possible that the residence time of ion at the treatment site will be short. In efforts to generate sustained release lithium salts, the hydrophobicity of the salt can be enhanced and made “lipid-like,” to, for example, lower the rate of ionization of the salt into lithium ions. For example, lithium chloride has a much faster rate of ionizing into lithium ions, than lithium stearate or lithium orotate. In that regard, the lithium salt can be that of a cholesterol derivative, or a long chain fatty acids or alcohols. Lipid complexed lithium salts of size less than 10 microns can also be effectively targeted to the hair follicles and “tethered” to the sebaceous glands, by hydrophobic-hydrophobic interactions.

In some embodiments, the organic lithium salt can be in the form of complexes with anionic compounds or anionic poly(amino acids) and other polymers. The complexes can be neutral, wherein all of the negative charges of the complexation agent are balanced by equimolar concentrations of Li ions. The complexes can be negatively charged, with lithium ions bound to an anionic polymer. The complexes can be in the form of nano-complexes, or micro-complexes, small enough to be targeted to the hair follicles. If the complexes are targeted to the dermis, the charged nature of the complexes will “tether” the complexes to the positively charged collagen. This mode of tethering holds the Li ions at the site of delivery, thereby hindering fast in-vivo clearance. Examples of negatively charged polymers that may be used are poly(acrylates) and its copolymers and derivatives thereof, hyaluronic acid and its derivatives, alginate and its derivatives, etc. In one variation, the anionic lithium complexes formed as described above can be further complexed with a cationic polymer such as chitosan, or polyethylimine form cell-permeable delivery systems.

The lithium salt can be that of a fatty acid, e.g., lithium stearate, thereby promoting absorption through skin tissues and extraction into the lipid compartments of the skin. In another example, the lithium salt of sebacic acid can be administered to the skin for higher absorption and targeting into structures of the skin, such as hair follicles.

The lithium salts may be inorganic lithium salts. Inorganic lithium salts for use in these embodiments include halide salts, such as lithium bromide, lithium chloride, lithium fluoride, or lithium iodide. In one embodiment, the inorganic lithium salt is lithium fluoride. In another embodiment, the inorganic lithium salt is lithium iodide. In certain embodiments, the lithium salts do not comprise lithium chloride. Other inorganic lithium salts for use in these embodiments include lithium borate, lithium nitrate, lithium perchlorate, lithium phosphate, or lithium sulfate.

The inorganic lithium salts may comprise the lithium salts of boric acid, hydrobromic acid, hydrochloric acid, hydrofluoric acid, hydroiodic acid, nitric acid, perchloric acid, phosphoric acid, or sulfuric acid.

Compositions containing one or more lithium compounds may be formulated with a pharmaceutically acceptable carrier (also referred to as a pharmaceutically acceptable excipients), i.e., a pharmaceutically-acceptable material, composition, or vehicle, such as a liquid or solid filler, diluent, solvent, an encapsulating material, or a complexation agent. In one embodiment, each component is “pharmaceutically acceptable” in the sense of being chemically compatible with the other ingredients of a pharmaceutical formulation, and biocompatible, when in contact with the biological tissues or organs of humans and animals without excessive toxicity, irritation, allergic response, immunogenicity, or other problems or complications, commensurate with a reasonable benefit/risk ratio. See, Remington: The Science and Practice of Pharmacy, 2005, 21st ed., Philadelphia, Pa.: Lippincott Williams & Wilkins; Rowe et al., eds., 2005, Handbook of Pharmaceutical Excipients, 5th ed., The Pharmaceutical Press and the American Pharmaceutical Association; Ash & Ash eds., 2007, Handbook of Pharmaceutical Additives, 3rd ed., Gower Publishing Company; Gibson ed., 2009, Pharmaceutical Preformulation and Formulation, 2nd ed., Boca Raton, Fla.: CRC Press LLC, each of which is incorporated herein by reference.

Suitable excipients are well known to those skilled in the art, and non-limiting examples of suitable excipients are provided herein. Whether a particular excipient is suitable for incorporation into a composition depends on a variety of factors well known in the art, including, but not limited to, the method of administration. For example, forms for topical administration such as a cream may contain excipients not suited for use in transdermal or intravenous administration. The suitability of a particular excipient depends on the specific active ingredients in the dosage form. Exemplary, non-limiting, pharmaceutically acceptable carriers for use in the lithium formulations described herein are the cosmetically acceptable vehicles provided in International Patent Application Publication No. WO 2005/120451, which is incorporated herein by reference in its entirety.

Lithium-containing compositions may be formulated to include an appropriate aqueous vehicle, including, but not limited to, water, saline, physiological saline or buffered saline (e.g., phosphate buffered saline (PBS)), sodium chloride for injection, Ringers for injection, isotonic dextrose for injection, sterile water for injection, dextrose lactated Ringers for injection, sodium bicarbonate, or albumin for injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, lanolin oil, lanolin alcohol, linoleic acid, linolenic acid and palm seed oil. Suitable water-miscible vehicles include, but are not limited to, ethanol, wool alcohol, 1,3-butanediol, liquid polyethylene glycol (e.g., polyethylene glycol 300 and polyethylene glycol 400), propylene glycol, glycerin, N-methyl-2-pyrrolidone (NMP), N,N-dimethylacetamide (DMA), and dimethyl sulfoxide (DMSO).

Lithium-containing compositions for use in the methods and systems disclosed herein may also be formulated with one or more of the following additional agents. Suitable antimicrobial agents or preservatives include, but are not limited to, alkyl esters of p-hydroxybenzoic acid, hydantoins derivatives, propionate salts, phenols, cresols, mercurials, phenyoxyethanol, benzyl alcohol, chlorobutanol, methyl and propyl p-hydroxybenzoates, thimerosal, benzalkonium chloride (e.g., benzethonium chloride), butyl, methyl- and propyl-parabens, sorbic acid, and any of a variety of quarternary ammonium compounds. Suitable isotonic agents include, but are not limited to, sodium chloride, glycerin, and dextrose. Suitable buffering agents include, but are not limited to, phosphate, glutamate and citrate. Suitable antioxidants are those as described herein, including ascorbate, bisulfite and sodium metabisulfite. Suitable local anesthetics include, but are not limited to, procaine hydrochloride, lidocaine and salts thereof, benzocaine and salts thereof and novacaine and salts thereof. Suitable suspending and dispersing agents include but are not limited to sodium carboxymethylcelluose (CMC), hydroxypropyl methylcellulose (HPMC), polyvinyl alcohol (PVA), and polyvinylpyrrolidone (PVP). Suitable emulsifying agents include but are not limited to, including polyoxyethylene sorbitan monolaurate, polyoxyethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

Soothing preparations, e.g., for topical administration, may contain sodium bicarbonate (baking soda), and coal tar based products. Formulations may also optionally contain a sunscreen or other skin protectant, or a waterproofing agent.

A product for application to the scalp or face may additionally be formulated so that it has easy rinsing, minimal skin/eye irritation, no damage to existing hair, has a thick and/or creamy feel, pleasant fragrance, low toxicity, good biodegradability, and a slightly acidic pH (pH less than 7), since a basic environment weakens the hair by breaking the disulfide bonds in hair keratin.

In particular embodiments, commercially available preparations of lithium can be used, such as, e.g., lithium gluconate, 8% lithium gluconate (Lithioderm™), approved for the treatment of seborrhoeic dermatitis (see, e.g., Dreno and Moyse, 2002, Eur J Dermatol 12:549-552; Dréno et al., 2007, Ann Dermatol Venereol 134:347-351 (abstract); and Ballanger et al., 2008, Arch Dermatol Res 300:215-223, each of which is incorporated by reference herein in its entirety); 8% lithium succinate (see, e.g., Langtry et al., 1996, Clinical and Experimental Dermatology 22:216-219; and Cuelenaere et al., 1992, Dermatology 184:194-197, each of which is incorporated by reference herein in its entirety); or 8% lithium succinate with 0.05% zinc sulfate (marketed in the U.K. as Efalith; see, e.g., Efalith Multicenter Trial Group, 1992, J Am Acad Dermatol 26:452-457, which is incorporated by reference herein in its entirety).

Certain lithium compounds are known to function as modulators of GSK3β (glycogen synthase kinase-3 beta). Other GSK3β modulators may be used as a physiologically active compound in accordance with the present compositions. Nonlimiting examples include: antibodies to GSK3β; 6-bromo-indirubin-3′-oxime (6-BIO); CHIR99021 (developed by Chiron, Emeryville, Calif.) (i.e., 6-[(2-{[4-(2,4-dichlorophenyl)-5-(4-methylimidazol-2-yl)pyrimidin-2-yl]amino}ethyl)amino]pyridine-3-carbonitrile); ARA014418 (AstraZeneca) (i.e., 4-(4-methoxybenzyl)-n′-(5-nitro-1,3-thiazol-2-yl)urea); TDZD-8 Noscira (Neuropharma) (i.e., 4-benzyl-2-methyl-1,2,4-thiadiazolidine-3,5-dione); “Compound 12” (i.e., 2-thio(3-iodobenzyl)-5-(1-pyridyl)-[1,3,4]-oxadiazole); and any combination thereof.

Still other GSK3β modulators may be used as a physiologically active compound in accordance with the present compositions. Further exemplary GSK3β modulators are listed below in Table 1.

TABLE 1

| Class or | | |
|---|---|---|
| Compound Name | Exemplary Compounds (if applicable) | Comments |
| Indirubin derivatives | 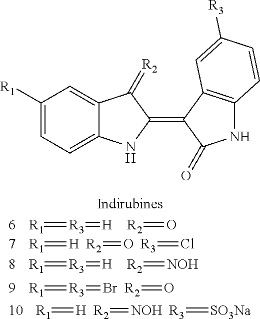 | 5- chloroindirubin (7) and indirubin 3′- monoxime (8) have better pharmacological properties and reduced toxicity |
| | 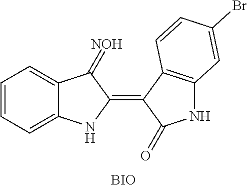 | |
| | 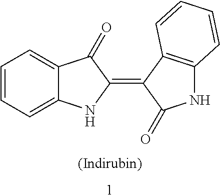 | |
| | 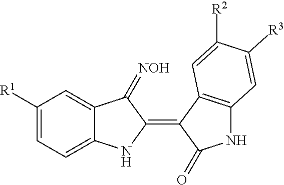 | |
| Kenpaullone and alsterpaullone | 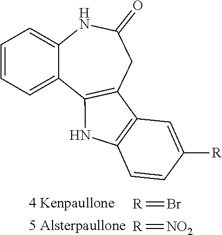 | |
| Purine Derivatives | 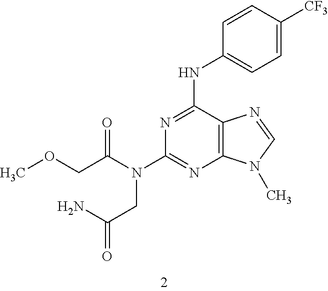 | Other Chiron compounds: CHIR 118637; CHIR 9803; CHIR 99021; CT 98023; CY 20026 |
| CHIR 9803 | 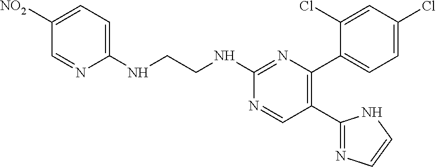 | |
| aminopyridine derivative | 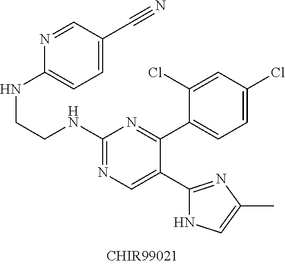 | |
| Core IS Maleimides- Bisindolylmaleimide derivatives of staurosporine | 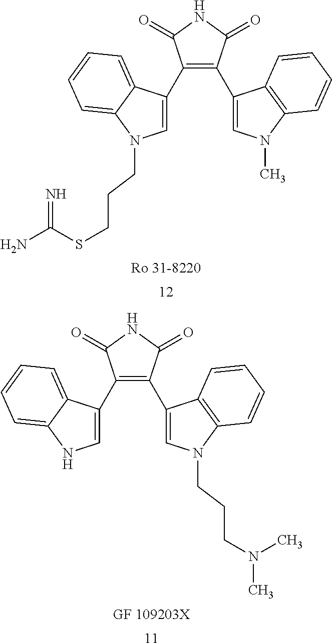 | |
| Core IS Maleimides | 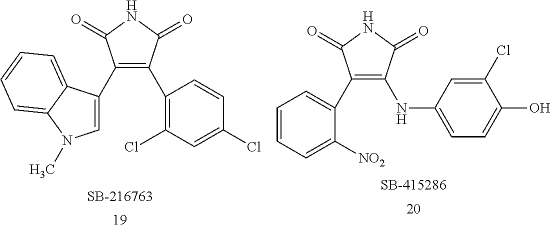 | |
| AR A014418 | 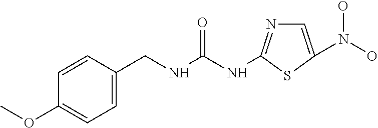 | |
| NNC 570558 | 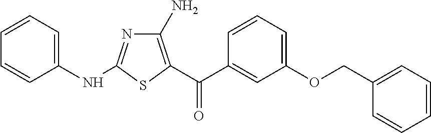 | |
| XD 4241 | Structure not known | Compound is |
| | | available for |
| | | licensing from |
| | | Xcellsyz, Ltd. |

The physiologically active compound for use in the present compositions can be a BMP inhibitor, such as the LDN-193189 small molecule (developed by Massachusetts General Hospital/Harvard); Dorsomorphin (pictured below)

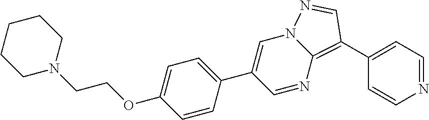

### or Dorsomorphin HCl; or, Noggin Protein (Stemgent, Cambridge, Mass.).

Other physiologically active compounds that may be used in the present compositions include Wnt modulators. For example, klotho is a protein that has been found to bind and inhibit Wnt interactions with Wnt-Receptor. See, e.g., Liu, H, et al., Science, Vol. 317. no. 5839, pp. 803-806, 10 Aug. 2007. Known Wnt agonists include 2-amino-4-(3,4-(methylenedioxy) benzylamino)-6-(3-methoxyphenyl)pyrimidine (see Osteoarthritis Cartilage. 2004 June; 12(6):497-505) and a “group of thiophene-pyrimidines” that were identified in an academic screen for drugs that induce pancreatic beta-cell expansion (see Proc Natl Acad Sci USA. 2009 Feb. 3; 106(5): 1427-32). These and any other Wnt modulators may be used in the present compositions.

Stem-cell signaling drug molecules may be encapsulated in matrices that are highly hydrophilic and charged, preferably linked to the dermis by covalent or ionic bonding to prevent the matrices from being cleared by phagocytosis, as part of the wound healing process.

The physiologically active compound can be a small molecule EGFR inhibitor, or metabolite thereof (e.g., a non-naturally occurring nitrogen-containing heterocycle of less than about 2,000 daltons, leflunomide, gefitinib, erlotinib, lapatinib, canertinib, vandetanib, CL-387785, PKI166, pelitinib, HKI-272, and HKI-357), EGF, an EGFR antibody (zalutumumab, cetuximab, IMC 11F8, matuzumab, SC 100, ALT 110, PX 1032, BMS599626, MDX 214, and PX 1041), a suppressor of the expression of a Wnt protein in the hair follicle or an inducer of expression of a Dkk1 protein (e.g., from lithium chloride, a molecule that synergizes with lithium chloride, the agonists 6-bromoindirubin-3′-oxime, deoxycholic acid, a pyrimidine derivative, antagonists quercetin, ICG-001, the purine derivative QS11, fungal derivatives PKF115-854 and CGP049090, and the organic molecule NSC668036), a modulator the retinoic acid signaling pathway (trans-retinoic acid, N-retinoyl-D-glucosamine, and seletinoid G), a modulator of the estrogen signaling pathway (e.g., 17β-estradiol and selective estrogen receptor modulators), a compound which modulates the ubiquitin-proteasome system, a compound which modulates cytokine signaling of Imiquimod or IL-1alpha, a modulator of melanocortin signaling, tyrosinase activity, apoptosis signaling, endothelin signaling, nuclear receptor signaling, TGFβ-SMAD signaling, bone morphogenetic protein signaling, stem cell factor signaling, androgen signaling, retinoic acid signaling, peroxisome proliferator-activated response receptor signaling, estrogen signaling, cytokine signaling, growth factor signaling, nonandrogenic hormone signaling, toll-like receptor signaling, and neurotrophin, neuroendocine signaling, and cytokine signaling, benzoyl peroxide, a photosenitizer (e.g., aminolevulinic acid), an interferon, dacarbazine, interleukin-2, imiquimod, or a promoter of the expression of the transcription factor MITF.

The phrase “small molecule EGFR inhibitor” refers to a molecule that inhibits the function of one or more EGFR family tyrosine kinases. Tyrosine kinases of the EGFR family include EGFR, HER-2, and HER-4 (see Raymond et al., Drugs 60 (Suppl. 1):15 (2000); and Harari et al., Oncogene 19:6102 (2000)). Small molecule EGFR inhibitors include, for example, gefitinib (Baselga et al., Drugs 60 (Suppl. 1):33 (2000)), erlotinib (Pollack et al., J. Pharm. Exp. Ther. 291:739 (1999)), lapatinib (Lackey et al., 92^{nd} AACR Meeting, New Orleans, abstract 4582 (2001)), canertinib (Bridges et al., Curr. Med. Chem. 6:825 (1999)), vandetanib (Wedge et al., Cancer Res. 62:4645 (2002)), CL-387785 (Discafani et al., Biochem. Pharmacol. 57:917 (1999)), PKI166 (Takada et al., Drug Metab. Dispos. 32:1272 (2004)), pelitinib (Torrance et al., Nature Medicine 6:1024 (2000)), HKI-272, HKI-357 (for HKI-272 and HM-357 see, for example, Greenberger et al., 11^{th} NCI-EORTC-AACR Symposium on New Drugs in Cancer Therapy, Amsterdam, abstract 388 (2000); Rabindran et al., Cancer Res. 64:3958 (2004); Holbro et al., Ann. Rev. Pharm. Tox. 44:195 (2004); Tsou et al., J. Med. Chem. 48:1107 (2005); and Tejpar et al., J. Clin. Oncol. ASCO Annual Meeting Proc. 22:3579 (2004)), and leflunomide (Kochhar et al., FEBS Lett. 334:161 (1993)). The structures for each of these compounds is provided below in Table 2.

TABLE 2
EGFR Inhibitors

| Drug | Structure |
|---|---|
| leflunomide | 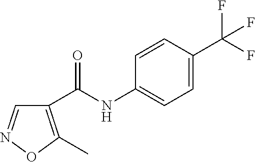 |
| Gefitinib | 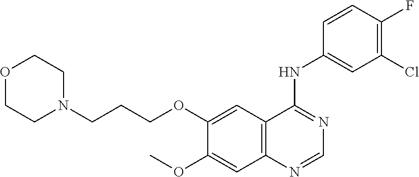 |
| Erlotinib | 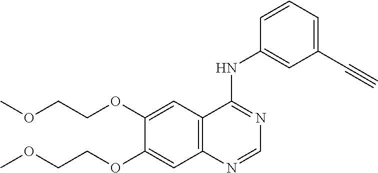 |
| Lapatinib | 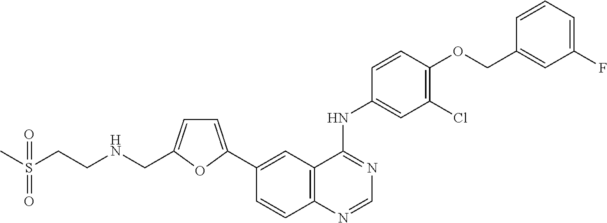 |
| Canertinib | 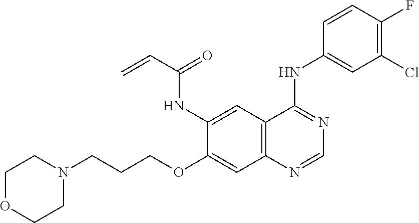 |
| Vandetanib | 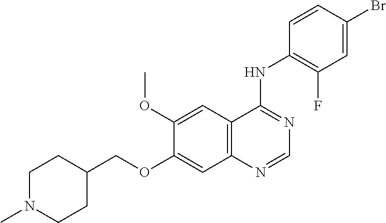 |
| CL-387785 | 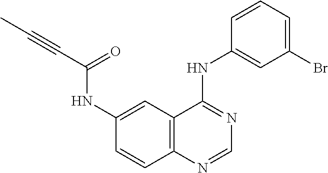 |
| PKI166 | 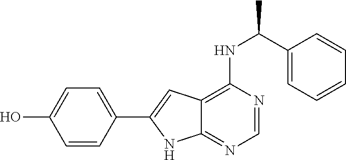 |
| Pelitinib | 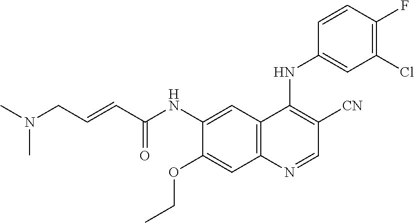 |
| HKI-272 | 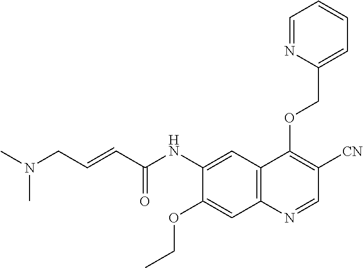 |
| HKI-357 | 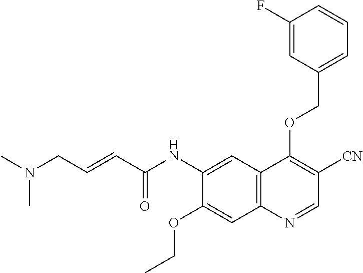 |

Small molecule EGFR inhibitors that can be used in the present compositions include anilinoquinazolines, such as gefitinib, erlotinib, lapatinib, canertinib, vandetanib, and CL-387785 and the other anilinoquinazolines disclosed in PCT Publication No. WO/2005/018677 and U.S. Pat. Nos. 5,747,498 and 5,457,105; quinoline-3-carbonitriles, such as pelitinib, BKI-272, and HKI-357, and the quinoline-3-carbonitriles disclosed in U.S. Pat. Nos. 6,288,082 and 6,002,008; pyrrolopyrimidines, such as PKI166, and the pyrrolopyrimidines disclosed in U.S. Pat. No. 6,713,474 and U.S. Patent Publication Nos. 20060211678, 20060035912, 20050239806, 20050187389, 20050165029, 20050153989, 20050037999, 20030187001, and 20010027197; pyridopyrimidines, such as those disclosed in U.S. Pat. Nos. 5,654,307 and 6,713,484; pyrazolopyrimidines, such as those disclosed in U.S. Pat. Nos. 6,921,763 and 6,660,744 and U.S. Patent Publication Nos. 20060167020, 20060094706, 20050267133, 20050119282, 20040006083, and 20020156081; isoxazoles, such as leflunomide; imidazoloquinazolines, pyrroloquinazolines, and pyrazoloquinazolines. Preferably, the small molecule EGFR inhibitor contains a heterobicyclic or heterotricyclic ring system. Each of the patent publications listed above is incorporated herein by reference.

A77 7628 refers to the active metabolite of leflunomide having the structure below.

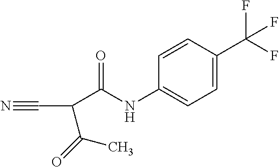

Useful antioxidants may include, without limitation, thiols (e.g., aurothioglucose, dihydrolipoic acid, propylthiouracil, thioredoxin, glutathione, cysteine, cystine, cystamine, thiodipropionic acid), sulphoximines (e.g., buthionine-sulphoximines, homo-cysteine-sulphoximine, buthionine-sulphones, and penta-, hexa- and heptathionine-sulphoximine), metal chelators (e.g, α-hydroxy-fatty acids, palmitic acid, phytic acid, lactoferrin, citric acid, lactic acid, and malic acid, humic acid, bile acid, bile extracts, bilirubin, biliverdin, EDTA, EGTA, and DTPA), vitamins (e.g., vitamin E, vitamin C, ascorbyl palmitate, Mg ascorbyl phosphate, and ascorbyl acetate), phenols (e.g., butylhydroxytoluene, butylhydroxyanisole, ubiquinol, nordihydroguaiaretic acid, trihydroxybutyrophenone), benzoates (e.g., coniferyl benzoate), uric acid, mannose, propyl gallate, selenium (e.g., selenium-methionine), stilbenes (e.g., stilbene oxide and trans-stilbene oxide), and combinations thereof.

Antioxidants that may be incorporated into the formulations of the invention include natural antioxidants prepared from plant extracts, such as extracts from aloe vera; avocado; chamomile; echinacea; ginko biloba; ginseng; green tea; heather; jojoba; lavender; lemon grass; licorice; mallow; oats; peppermint; St. John's wort; willow; wintergreen; wheat wild yam extract; marine extracts; and mixtures thereof.

The total amount of antioxidant included in the formulations can be from 0.001% to 3% by weight, preferably 0.01% to 1% by weight, in particular 0.05% to 0.5% by weight, based on the total weight of the formulation.

The composition that is applied to the target area may include one or more antihistamines. Exemplary antihistamines include, without limitation, Ethanolamines (e.g., bromodiphenhydramine, carbinoxamine, clemastine, dimenhydrinate, diphenhydramine, diphenylpyraline, and doxylamine); Ethylenediamines (e.g., pheniramine, pyrilamine, tripelennamine, and triprolidine); Phenothiazines (e.g., diethazine, ethopropazine, methdilazine, promethazine, thiethylperazine, and trimeprazine); Alkylamines (e.g., acrivastine, brompheniramine, chlorpheniramine, desbrompheniramine, dexchlorpheniramine, pyrrobutamine, and triprolidine); piperazines (e.g., buclizine, cetirizine, chlorcyclizine, cyclizine, meclizine, hydroxyzine); Piperidines (e.g., astemizole, azatadine, cyproheptadine, desloratadine, fexofenadine, loratadine, ketotifen, olopatadine, phenindamine, and terfenadine); and Atypical antihistamines (e.g., azelastine, levocabastine, methapyrilene, and phenyltoxamine). Both non-sedating and sedating antihistamines may be employed. Non-sedating antihistamines include loratadine and desloratadine. Sedating antihistamines include azatadine, bromodiphenhydramine; chlorpheniramine; clemizole; cyproheptadine; dimenhydrinate; diphenhydramine; doxylamine; meclizine; promethazine; pyrilamine; thiethylperazine; and tripelennamine.

Other suitable antihistamines include acrivastine; ahistan; antazoline; astemizole; azelastine; bamipine; bepotastine; bietanautine; brompheniramine; carbinoxamine; cetirizine; cetoxime; chlorocyclizine; chloropyramine; chlorothen; chlorphenoxamine; cinnarizine; clemastine; clobenzepam; clobenztropine; clocinizine; cyclizine; deptropine; dexchlorpheniramine; dexchlorpheniramine maleate; diphenylpyraline; doxepin; ebastine; embramine; emedastine; epinastine; etymemazine hydrochloride; fexofenadine; histapyrrodine; hydroxyzine; isopromethazine; isothipendyl; levocabastine; mebhydroline; mequitazine; methafurylene; methapyrilene; metron; mizolastine; olapatadine; orphenadrine; phenindamine; pheniramine; phenyltoloxamine; p-methyldiphenhydramine; pyrrobutamine; setastine; talastine; terfenadine; thenyldiamine; thiazinamium; thonzylamine hydrochloride; tolpropamine; triprolidine; and tritoqualine.

Antihistamine analogs may also be used. Antihistamine analogs include 10-piperazinylpropylphenothiazine; 4-(3-(2-chlorophenothiazin-10-yl)propyl)-1-piperazineethanol dihydrochloride; 1-(10-(3-(4-methyl-1-piperazinyl)propyl)-10H-phenothiazin-2-yl)-(9CI) 1-propanone; 3-methoxycyproheptadine; 4-(3-(2-Chloro-10H-phenothiazin-10-yl)propyl)piperazine-1-ethanol hydrochloride; 10,11-dihydro-5-(3-(4-ethoxycarbonyl-4-phenylpiperidino)propylidene)-5H-dibenzo(a,d)cycloheptene; aceprometazine; acetophenazine; alimemazin (e.g., alimemazin hydrochloride); aminopromazine; benzimidazole; butaperazine; carfenazine; chlorfenethazine; chlormidazole; cinprazole; desmethylastemizole; desmethylcyproheptadine; diethazine (e.g., diethazine hydrochloride); ethopropazine (e.g., ethopropazine hydrochloride); 2-(p-bromophenyl-(p′-tolyl)methoxy)-N,N-dimethyl-ethylamine hydrochloride; N,N-dimethyl-2-(diphenylmethoxy)-ethylamine methylbromide; EX-10-542A; fenethazine; fuprazole; methyl 10-(3-(4-methyl-1-piperazinyl)propyl)phenothiazin-2-yl ketone; lerisetron; medrylamine; mesoridazine; methylpromazine; N-desmethylpromethazine; nilprazole; northioridazine; perphenazine (e.g., perphenazine enanthate); 10-(3-dimethylaminopropyl)-2-methylthio-phenothiazine; 4-(dibenzo(b,e)thiepin-6(11H)-ylidene)-1-methyl-piperidine hydrochloride; prochlorperazine; promazine; propiomazine (e.g., propiomazine hydrochloride); rotoxamine; rupatadine; Sch 37370; Sch 434; tecastemizole; thiazinamium; thiopropazate; thioridazine (e.g., thioridazine hydrochloride); and 3-(10,11-dihydro-5H-dibenzo(a,d)cyclohepten-5-ylidene)-tropane.

Other compounds that may be used in the present compositions include AD-0261; AHR-5333; alinastine; arpromidine; ATI-19000; bermastine; bilastin; Bron-12; carebastine; chlorphenamine; clofurenadine; corsym; DF-1105501; DF-11062; DF-1111301; EL-301; elbanizine; F-7946T; F-9505; HE-90481; HE-90512; hivenyl; HSR-609; icotidine; KAA-276; KY-234; lamiakast; LAS-36509; LAS-36674; levocetirizine; levoprotiline; metoclopramide; NIP-531; noberastine; oxatomide; PR-881-884A; quisultazine; rocastine; selenotifen; SK&F-94461; SODAS-HC; tagorizine; TAK-427; temelastine; UCB-34742; UCB-35440; VUF-K-8707; Wy-49051; and ZCR-2060.

Still other compounds that may be used in the present compositions are described in U.S. Pat. Nos. 3,956,296; 4,254,129; 4,254,130; 4,282,233; 4,283,408; 4,362,736; 4,394,508; 4,285,957; 4,285,958; 4,440,933; 4,510,309; 4,550,116; 4,692,456; 4,742,175; 4,833,138; 4,908,372; 5,204,249; 5,375,03; 5,578,610; 5,581,011; 5,589,487; 5,663,412; 5,994,549; 6,201,124; and 6,458,958.

The compositions that are applied to the target area may include an antimicrobial agent. Useful antimicrobial agents include, without limitation, benzyl benzoate, benzalkonium chloride, benzoic acid, benzyl alcohol, butylparaben, ethylparaben, methylparaben, propylparaben, camphorated metacresol, camphorated phenol, hexylresorcinol, methylbenzethonium chloride, cetrimide, chlorhexidine, chlorobutanol, chlorocresol, cresol, glycerin, imidurea, phenol, phenoxyethanol, phenylethylalcohol, phenylmercuric acetate, phenylmercuric borate, phenylmercuric nitrate, potassium sorbate, sodium benzoate, sodium proprionate, sorbic acid, and thiomersal.

The antimicrobial may be from about 0.05% to 0.5% by weight of the total composition, except for camphorated phenol and camphorated metacresol. For camphorated phenol, the preferred weight percentages are about 8% to 12% camphor and about 3% to 7% phenol. For camphorated metacresol, the preferred weight percentages are about 3% to 12% camphor and about 1% to 4% metacresol.

The compositions that are applied to the target area may include an anti-inflammatory agent. Useful antiinflammtory agents include, without limitation, Non-Steroidal Anti-Inflammatory Drugs (NSAIDs) (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), and corticosteroids (e.g., alclometasone dipropionate, amcinonide, betamethasone dipropionate, betamethasone valerate, clobetasol propionate, desonide, desoximetasone, dexamethasone, diflorasone diacetate, flucinolone acetonide, flumethasone, fluocinonide, flurandrenolide, halcinonide, halobetasol propionate, hydrocortisone butyrate, hydrocortisone valerate, methylprednisolone, mometasone furoate, prednisolone, or triamcinolone acetonide).

The compositions that are applied to the target area may include a nonsteroidal immunosuppressant. Suitable immunosuppressants include cyclosporine, tacrolimus, rapamycin, everolimus, and pimecrolimus.

The cyclosporines are fungal metabolites that comprise a class of cyclic oligopeptides that act as immunosuppressants. Cyclosporine A is a hydrophobic cyclic polypeptide consisting of eleven amino acids. It binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T cell-dependent immune response by inhibiting antigen-triggered signal transduction. This inhibition decreases the expression of proinflammatory cytokines, such as IL-2.

Many different cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Application Publication No. 2002/0132763 A1). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH-Cs (209-825), Allo-Thr-2-Cs, Norvaline-2-Cs, D-Ala(3-acetylamino)-8-Cs, Thr-2-Cs, and D-MeSer-3-Cs, D-Ser(O—CH₂CH₂—OH)-8-Cs, and D-Ser-8-Cs, which are described in Cruz et al., Antimicrob. Agents Chemother. 44:143 (2000).

Tacrolimus and tacrolimus analogs are described by Tanaka et al. (J. Am. Chem. Soc., 109:5031 (1987)) and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus is the 33-epi-chloro derivative of the macrolactam ascomyin. Pimecrolimus structural and functional analogs are described in U.S. Pat. No. 6,384,073.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

The compositions that are applied to the target area may include a retinoid. Useful retinoids include, without limitation, 13-cis-retinoic acid, 9-cis retinoic acid, all-trans-retinoic acid, etretinate, acitretin, retinol, retinal, tretinoin, alitretinoin, isotretinoin, tazarotene, bexarotene, and adapelene.

In certain embodiments, the compositions that are applied to the target area may include a channel opener. Useful channel openers include, without limitation, minoxidil, diazoxide, and phenyloin.

In other embodiments, an anti-androgen can be used in the compositions that are applied to the target area. Useful anti-androgens include, without limitation, finasteride, flutamide, diazoxide, 11alpha-hydroxyprogesterone, ketoconazole, RU58841, dutasteride, fluridil, QLT-7704, and anti-androgen oligonucleotides.

In certain embodiments, the compositions that are applied to the target area may include an antibiotic. Useful antibiotics include, without limitation, penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, erythromycin, azithromycin, clarithromycin, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, sitafloxacin, metronidazole, daptomycin, garenoxacin, ramoplanin, faropenem, polymyxin, tigecycline, AZD2563, and trimethoprim.

Growth factors and growth factor antagonists can also be used in the compositions that are applied to the target area.

The composition may comprise an active ingredient for stimulating hair growth. Nonlimiting examples include monoxidil, finasteride, dutasteride, a copper peptide, saw palmetto extract, black cohosh, caffeine, or any combination thereof.

The composition that is applied to the target area may comprise a biological material. For example, DNA, RNA, cells (such as stem cells, nurse cells, keratinocytes), cellular components (collagen, elastin, cytoskeletal components, keratin), proteins, skin graft material, antibodies, viruses, or any other living or quasi-living material or product of a living system. As described more fully below, the composition, whether a biological material or another type of material may be applied substantially directly to the target area, and may even be applied substantially into the injured portion thereof.

The composition may comprise protective covering or sealant. Polymers, skin grafts, synthetic skin, biological glues, or any other material that is capable of forming a protective layer or seal at the injured target area is contemplated. In certain embodiments, the application of a composition to the injured target area may include the application of a material or compound of any other type described herein, sequentially followed by the application of a protective covering or sealant.

A biocompatible, synthetic skin substitute may be placed on a portion of tissue that has been injured in accordance with the present disclosure, especially if the wound is deep, covers large area, and has been bulk ablated. This process can help minimize or prevent the rapid wound contraction that occurs after loss of a large area of tissue, frequently culminating in scar tissue formation and loss of skin function. The biocompatible synthetic skin substitute may be impregnated with depots of slow releasing stem cell signaling molecules to channel the proliferating stem cell population toward hair follicle germ formation. This method of treatment may enable treating a large bald area on the scalp in one session at the treatment clinic. Other molecules may be co-eluted at the site through the skin substitute, such as anesthetics and antibiotics, to prevent further pain and minimization of infection. The skin substitute containing drug, as described herein, may also be pre-cooled and applied to the wound to provide a feeling of comfort to the patient. This mode of drug application may prevent the drug from being cleared away from the wound site, as the wound heals.

It is also envisioned that a compound absorbing light at specific wavelengths (e.g., between 1000-1600 nm may be included in a composition according to the present disclosure for the purpose of efficient channeling of light to heat energy. This method of channeling energy may cause micro-zones of thermal injury within the body surface. The compound may be delivered to the body surface homogenously in the treatment zone, then subsequently irradiated, for example, with a non-ablative laser, to efficiently capture the vibrational energy of the beam. This method may result in evenly distributed and deep thermal injury, without causing tissue vaporization.

Any other material or compound that may be useful for promoting or aiding in a desired outcome, including regeneration, remodeling, resurfacing, restoration, follicular neogenesis, neocollagenesis, stem cell recruitment, activation, or differentiation, reepitheliazation, wound healing, or any other desired biological or physical modification, may be applied to the target area in accordance with the present disclosure. Other suitable materials are described in WO/2008/143928, which is incorporated herein by reference in its entirety. Other materials of interest may include pigments, inks, dyes, or toxins (including neurotoxins, such as botulinum toxin). For example, a dye may be to designate locations already treated (i.e., for monitoring purposes); such dyes may be degradable over time or otherwise invisible under normal lighting conditions but visible under ultraviolet light or when activated in any other fashion.

The composition may be applied as a fluid (e.g., a liquid, gel, or gas) or as a solid (e.g., as a particulate material). The composition may be applied to the body surface or to some location beneath the body surface (e.g., into the tissue beneath the surface). The propulsion of drug-containing particles into a body surface—in particular, skin—is described at length PCT/US08/11979, the contents of which are incorporated herein in their entirety. The composition may comprise components that cause gelling or hardening of the composition. The gelling or hardening may occur as a result of a reaction between two or more components within the composition (as discussed more fully herein, in such embodiments the application of the composition may include the mixing of reactive components that form a gel following application of the composition to the target area). Exemplary compositions that form gels are disclosed infra. In other embodiments, the composition may be accelerated and “shot” in a narrow stream into part or all of the target area, much in the manner of transdermal particle injection systems or “gene guns” that are used to deliver a narrow stream of material through the stratum corneum layer of skin.

Compositions for topical administration for preferably local but also possible systemic effect, include emulsions, solutions, suspensions, creams, gels, hydrogels, ointments, dusting powders, dressings, elixirs, lotions, suspensions, tinctures, pastes, powders, crystals, foams, films, aerosols, irrigations, sprays, suppositories, sticks, bars, ointments, bandages, wound dressings, microdermabrasion or dermabrasion particles, drops, and transdermal or dermal patches. The topical formulations can also comprise micro- and nano-sized capsules, liposomes, micelles, microspheres, microparticles, nanosystems, e.g., nanoparticles, nano-coacervates and mixtures thereof. See, e.g., International Patent Application Publication Nos. WO 2005/107710, published Nov. 17, 2005, and WO 2005/020940, published Mar. 10, 2005, each of which is incorporated herein by reference in its entirety. In one embodiment, the nano-sized delivery matrix is fabricated through a well-defined process, such as a process to produce lithium encapsulated in a polymer. In another embodiment, a drug-releasing compound is spontaneously assembled in aqueous solutions, such as in liposomes and micelles.

The modality for injuring the target area may also be used to apply the composition to the target area. For example, a needle may be used to injure a target area and as a composition-delivery conduit. The propulsion of drug-containing particles into a body surface invokes a microdermabrasion model to injure the target area while simultaneously delivering a drug-containing composition (see PCT/US08/11979). A high-pressure jet of fluid (with or without abrasive particles within the fluid) may be used to injure a target area, and if the fluid contains a composition, then injury and application of a composition may be performed simultaneously. Water jet technology, for example, was developed in the 1950's and may be used to cut or puncture soft or hard materials (see, for example, Flow International Corporation, Kent, Wash.). Any other approach for using the injuring modality for applying a composition to a target area may be used.

The composition that is applied to the target area may allow for the delivery of physiologically active material to the target area immediately or after a period of delay. For example, the composition may comprise a physiologically active compound that will contact the target area as soon as the composition is applied and/or may comprise a physiologically active compound that is encapsulated within a degradable material so that the compound does not contact the target area until the degradable material breaks down or is worn away in situ. In this and other embodiments, the period of delay may be minutes, hours, or days, for example, about 10 minutes, about 30 minutes, about one hour, about two hours, about three hours, about six hours, about eight hours, about 12 hours, about 24 hours, about 36 hours, about two days, about three days, about one week, about two weeks, about three weeks, or any other desired period of delay. Once delivery of the physiologically active material has commenced, the rate of release may have any desired profile, such as constant or ascending. Those of ordinary skill in the pharmaceutical arts will readily appreciate available methods for achieving a desired release profile. For example, a plurality of tiny “pills” that individually comprise a dose of a drug and a wall may be included in the composition that is delivered to the target area, wherein the plurality of tiny pills comprises at least two separate populations of pills, wherein the respective walls of the pills in the first population are thicker than the respective walls of the pills in the second population, and wherein the respective doses of drug within the pills in the first population are greater than the respective doses of drug within the'pills in the second population in order to provide for an increasing release rate. Procedures for manufacturing tiny pills are disclosed in U.S. Pat. Nos. 4,434,153; 4,721,613; 4,853,229; 2,996,431; 3,139,383 and 4,752,470.

The preparation of various pharmaceutical formulations and exemplary components thereof, including controlled and extended release formulations, topical formulations, emulsifying excipients for use in formulations, gelling agents, hydrocolloids, cross-linking agents, and plasticizers are disclosed in WO 2008/143928, the entire contents of which are incorporated herein by reference.

Any gel or other matrix may be used pursuant to the present compositions. Gels or other matrices that optionally comprise one or more physiologically active compounds may be delivered into “micro”-channels (hereafter, “channels”) created by such skin disruption devices as fractional lasers, microneedle flat arrays or rollers, or any other device that creates channels in the body surface. For example, when the body surface is skin, the channel may extend through the stratum corneum, epidermis, and partially or fully into the dermis.

The matrices may be delivered as a drug-containing liquid into the channels, for example, by a device that can deliver precise volumes. In addition to the drug, the liquid, or the “vehicle” may contain a polymer, or a combination of polymers that either are thermoreversible, or viscosity enhancing, or act as ionic supports for the drug. By definition, “thermoreversible” means that aqueous solutions of the polymer display viscoelastic properties that are “reversed” or opposite to what is typically observed in fluids when they are heated or cooled. As an example, aqueous solutions of Polyethylene oxide-co-polypropylene oxide-co-polyethylene oxide (PEO-PPO-PEO) polymers have very low viscosity when cooled, slowly forming a hydrogel when warmed up to physiological temperatures. This property can be modulated by varying the concentration of the polymer and/or varying the ratio of the PEO/PPO segments. Thus, the temperature at which the polymer in solution reaches gelation is lower when the concentration of the polymer is higher. In an application of this property to current embodiment, a cold low viscosity solution can be “streamed” into the channels, which would then form a physically crosslinked gel upon warming to body temperature. By definition, a “physical cross-link” is not a covalent link, but is based on hydrogen bonds, ionic interactions and molecular entanglement of polymer chains. Delivery of a cold solution also provides a comfortable or soothing “feel” to the patient. A physically crosslinked solution is not a permanent crosslink, and generally diffuses or clears from the site by absorption. These types of polymer vehicles are preferred over permanently crosslinked polymers or hydrogels due to their biocompatibility with surrounding cells and tissues. Permanently crosslinked gels are biocompatible only if they are bioabsorbable by hydrolysis or proteolysis.

The polymer matrix that is delivered into the channels may comprise a biodegradable polymer than is degradable by hydrolysis or proteolysis. In addition, the biodegradable polymer may have difunctional crosslinkable groups that react to form covalent crosslinks in order to form a hydrogel. Hydrogel formation can be through use of redox reactive groups, or photoreactive groups or crosslinking through reaction between a highly reactive electrophile and nucleophile. For this embodiment, crosslinking initiators need to be part of the matrix. Crosslinking by polymerization can be initiated by a redox initiator, or a photoinitiator. UV light, visible light or infrared can be used to initiate the crosslinking reaction to form the hydrogel. In one embodiment, a laser or other form of electromagnetic energy used to create the channels can be used to crosslink the hydrogel.

The “biodegradable polymer” disclosed above may contain water-soluble moieties such as polyethylene oxide, chain extended by lactates, glycolates and end-capped with crosslinkable moieties such as acrylates. The biodegradable polymer may be thermoreversible, wherein the polymer is highly fluid when cold and viscous at higher temperatures, but is biodegradable and crosslinkable. An example of this type of polymer is acrylate-lactate-PEO-PPO-PEO-lactate-acrylate. In another embodiment, the crosslink density or mesh size of the hydrogel can be modulated by using polymers of varying functionalities. For example, a four-armed polymer core can be used to achieve a hydrogel with a smaller mesh size than one achieved with a difunctional polymer core.

In another embodiment of a crosslinkable, biodegradable hydrogel, a biopolymer that reacts with components in tissue can be used to form a hydrogel.

Physiologically active compounds that are contained within physically crosslinked gels as described above are released from the matrix. The rate of release from this matrix is primarily controlled by the properties of the drug, i.e., if the molecular weight of the drug is much less than the pore size of the matrix. Typically, this is the case for small molecule drugs, with release rates being governed by the drug's solubility in water. A hydrophobic drug can be incorporated into an aqueous gel as microparticulate drug, with its release from the matrix rate-limited by the rate of dissolution of the drug in water. A hydrophilic drug, if not bound to the matrix by an interaction such as an ionic interaction, would be released from a physically crosslinked matrix very quickly, depending upon the molecular weight of the drug. For example, this type of matrix would be more appropriate for a hydrophilic protein than a hydrophilic small molecule. To slow down release of an ionic hydrophilic drug, use of a matrix that can ionically bind the drug, is a favorable option. Additionally, the hydrophilic drug such as a lithium salt, can be incorporated into solid lipid nanoparticles, then suspended in a viscous liquid like a cream, gel or emulsion.

Drugs that are small molecular and hydrophilic may be encapsulated into biodegradable microspheres, and then incorporated into a gel for delivery into a channel. This method can significantly slow down the diffusion of the drug from the site. The rate of release of the drug from the microspheres can be modulated by choice of the polymer. For example, a PLG polymer of molecular weight 12,000 Daltons releases drug at a much slower rate than a PLG polymer of molecular weight 30,000 Daltons. In another example, a PLG polymer with acid end groups release drug at faster rate than a PLG polymer with ester end groups. In another example, polylactic acid (PLA) releases drug very slowly, due to its low rate of hydrolytic degradation. Thus, the rate of drug release can be modulated appropriately by choice of the polymer used to encapsulate the drug. This approach can be used in a similar fashion for hydrophobic drugs.

In some embodiments, a drug-containing polymer solution is delivered into the channels using a delivery device and the solvent used to dissolve the biodegradable polymer diffuses out into surrounding tissue, leaving behind substantially solid columns of drug-containing matrix. An example of this type of matrix is PLG polymer+drug dissolved in a low molecular weight polyethylene glycol (PEG 300) as the solution to be delivered into the channels. After administration, the water soluble PEG300 diffuses into the surrounding tissue, leaving behind what is effectively a sustained release drug delivery system.

In another embodiment, the drug is encapsulated in a cavitrant molecule such as cyclodextrin, and derivatives thereof.

Application of the composition “to” the first target area is intended to embrace application of the composition onto the body surface at the location of the target area, application of the composition within the body surface at the location of target area, application of the composition onto or within the body surface at the location of the target area and also onto or within the body surface at one or more locations that are substantially adjacent to the target area. Application of a composition to one target area at the same time as application of a composition to a further target area, to all or part of the rest of the portion of the body surface, or both, are also intended to be embraced by the application of the composition “to” the target area.

The application of the composition to the target area may be accomplished by any method that contacts the composition with the target area. For example, the composition may be sprayed, dripped, painted, propelled, misted, or injected in order to apply it to the target area. The application of the composition to the target area may be topical, may be to some location at the target area that is interior to the body surface, or both. In some embodiments, the composition is a fluid that is sprayed onto the target area. In other embodiments, the composition is sprayed, propelled, or injected into the injured target area, which may include contacting only the injured portion of the target area with the composition, contacting only the target area with the composition, contacting substantially only the target area with the composition (i.e., wherein only incidental amounts of composition are applied to areas of the body surface beyond the target area), or contacting the target area and one or more adjacent areas of the body surface with the composition.

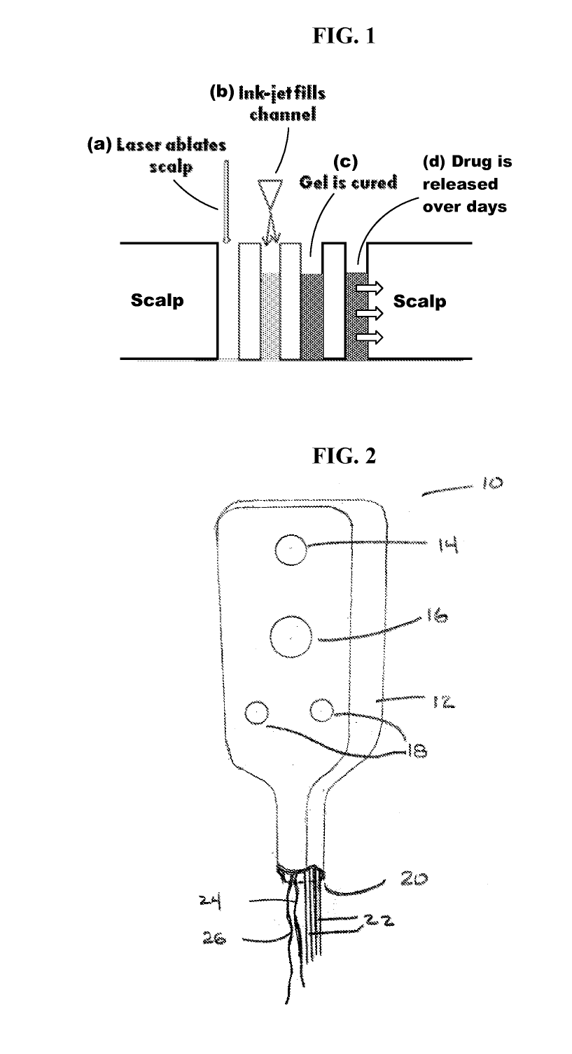

When the target area is injured by removing a column of tissue to form a channel, the composition may be applied substantially directly into the channel. The application of the composition “substantially directly” into the channel refers to the delivery of one or more aliquots of composition into the channel that may or may not include the delivery of an amount of composition to the target area outside of the channel, to one or more adjacent area of the body surface, or both. Depending on the chosen means for applying the composition substantially directly into the channel, the composition may be precisely delivered into the channel with no or only incidental amounts of composition being delivered outside of the channel. For example, inkjet-type technology may be used for precise application of the composition into the channel, and in this manner, a composition containing a physiologically active compound, a biological material, or any other desired agent may be introduced into the body surface at a desired location. The delivery of cells via inkjet printer has been reported (see, e.g., S. Webb, “Life in Print. Cell by cell, ink-jet printing builds living tissues”. Science News, Vol. 173, Jan. 26, 2008), and such technology may be used for the precise administration of biological material, physiologically active compound, or the like into an injury in a target area in accordance with the present disclosure. In some embodiments, the composition that is applied substantially directly into a channel at a target area may be a fluid that forms a gel in situ. A composition of this variety may release a physiologically active compound into the target area at a desired release rate, e.g., an immediate release or a controlled rate of release over time. FIG. 1 illustrates (a) the use of a fractional laser to form a hole in human skin, after which (b) the hole is filled with a highly viscous drug-containing gel via an ink-jet precision fill device. At step (c), body heat or other external factors crosslink the gel into a stable drug-releasing matrix, and (d) drug is released from the matrix over time.

Thus, a drug containing gel matrix can be delivered into the holes created by what is tantamount to a fractional FTE modality (e.g., laser, micro needles, miniature punch biopsy needles, and the like). Poly-phasic biocompatible gels such as pluronic “F-127” can be produced in a highly viscous drug contacting solution or emulsion. At room temperature, these solutions can be readily delivered via ink-jet or by precision industrial “micro-fill” technology. MicroFab, Inc. of Plano, Tex. provides a piezo-based high-speed fluidic delivery systems that can accurately deliver these volumes (e.g., ⅓ mm³ per hole). Once the drug contacting pluronic solution is delivered into the hole, body heat permanently changes the highly viscous solution into a stable gel. The gel may then release drug over time as the holes heal. In accordance with the present disclosure, drug may be released over about 12 hours to about 20 days, about 1 day to about 10 days, or about 3 days to about 7 days, or over other longer or shorter periods of time, as desired. Other highly viscous drug contacting macromonomeric biocompatible solutions (examples described supra) can be cross-linked into a stable drug releasing hydrogel. For cross-linking to occur, the polymer must have crosslinkable moieties such as acrylates. Crosslinking can be achieved by incorporating a photoinitiator such as Darocure or Irgacure and initiated by light (UV light, visible light, laser light). Crosslinking can also be achieved using a GRAS redox initiator, wherein the crosslinking mechanism does not involve heat, or light, but an oxidation reduction reaction.

The step of applying “a composition” to the target area may include the application of two or more compositions, and the compositions may respectively be applied using a desired modality. For example, a first composition may be applied to the target area in the form of a fluid that is applied substantially directly into a channel that was formed at the target area, and a second composition may be a protective covering or seal that is applied onto the target area and over the injury to protect or seal the first composition within the channel or otherwise shield the injury from the ambient environment. In such instances, the first composition may be applied using inkjet-type technology, and the second composition may be applied using conventional spray technology. All combinations of composition types and application modalities are contemplated as being embraced by the present disclosure.

Following the injuring of the first target area on the body surface and the optional application of a composition to the injured first target area, a further target area is selected on the body surface. The further target area has a preselected geometry with respect to the first target area. The “preselected geometry” may be based on a set of coordinates that collectively form a pattern, wherein the first target area and the second target area respectively represent successive coordinates within the pattern. For example, the pattern from which the preselected geometry is derived may be based upon a rectilinear grid, a curvilinear grid, a tessellation, a Fibonacci sequence, or any other regular, semiregular, or irregular arrangement of coordinates (points) or shapes. Thus, the first target area may represent a first coordinate or shape within the pattern, and the further target area will constitute the succeeding coordinate or shape with the same pattern. The “preselected geometry” need not be selected from an ordered array of coordinates or shapes, and the further target area may in fact be assigned through a randomized selection; in such instances, the first target area may represent a first coordinate or shape, and the further target area will constitute a second coordinate having a spatial relationship relative to the first target area that is randomly assigned, i.e., is “predetermined” in the sense that it was known beforehand that its spatial relationship to the first target area would be randomly assigned.

The selection of the further target area may be performed by a human controller, or may be performed by computerized system having the appropriate software. A human controller may provide initial instructions to a computer in order to identify a particular pattern or other basis for the preselected geometry (for example, the human controller may select a rectilinear grid as the pattern upon which the determination of the further target area or areas is based), and a computerized system may select the further target areas by proceeding in accordance with the initial instructions that were provided by the human controller. Thus, the computerized system and software may be capable of proceeding according to any of a number of different preloaded patterns, and may only require the input of a human controller as to which pattern should be used in order to commence the selection of a further target area or areas. One of ordinary skill in the art will readily appreciate how to obtain or create software that includes the instructions necessary for selecting one or more further target areas based on an ordered array or in accordance with a randomized selection.

Next, the further target area is injured. The injuring of the further target area may be performed using the same or comparable criteria as those described above with respect to the injuring of the first target area. Thus, the injuring of the further target area may be any modality that is suitable for inducing regeneration, remodeling, resurfacing, restoration, follicular neogenesis, neocollagenesis, stem cell recruitment, activation, or differentiation, reepitheliazation, wound healing, or any other desired biological or physical modification. Likewise, the injury may be induced by any mechanical, chemical, energetic, sound- or ultrasound-based, or electromagnetic means. The entire description provided above with respect to the injuring of the first target area is applicable to the injuring of the further target area. However, the mode of injuring of the further target area, or indeed of any subsequent further target area, may be the same as or different than the mode of injury that was used with respect to the preceding target area(s). For example, the present methods and systems may operate in accordance with a protocol that alternates or otherwise varies the type of injury that is used with respect to succeeding target areas. The protocol may establish a pattern with respect to sequential target areas (e.g., injury type A, followed by B, followed by C, followed by A, etc.), or may randomly assign an injury type to a particular target area (e.g., injury type A, followed by injury type D, followed by injury type B, followed by injury type B, and the like), wherein the injury types may be selected from a preselected set. The preselected set of injuries may correspond to the type of body surface to be treated (for example, if the body surface is the skin of a subject's scalp, then injury types that are suitable for use on the scalp may populate the set).

A composition may optionally be applied to the injured further target area. Any composition that is applied to the injured further target area may be the same as or different than the composition that is applied to the first target area. The parameters of the application of a composition to the injured further target area (e.g., the timing of the application relative to the injury, the type of composition, the mode of application, and the like) may be determined using the same criteria described above with respect to the application of a composition to the first injured target area. Thus, the entire description provided above with respect to the application of a composition to the first target area is pertinent to the application of a composition to the further target area.

Furthermore, the steps of selecting a further target area on the body surface, wherein the further target area has a preselected geometry with respect to the preceding target area; injuring the further target area; and optionally applying a composition to the injured further target area, may be performed iteratively to give rise to one or more additional target areas that are injured and optionally contacted with a composition. Collectively, the target areas may form a pattern upon or relative to the body surface. As described above, the pattern from which the preselected geometry of each successive target area is derived may be based upon a rectilinear grid, a curvilinear grid, a tessellation, a Fibonacci sequence, or any other regular, semiregular, or irregular arrangement of coordinates (points) or shapes, or may represent the results of a randomized selection of the preselected geometry.

In another aspect, systems for treating a body surface are provided comprising a traumatizer for inducing injury to a first target area at the body surface; an applicator for delivering a composition to the first target area; wherein the traumatizer, the applicator, or both are under the operative control of a general purpose digital computer; and wherein the computer is configured for selecting a further target area on the body surface having a preselected geometry with respect to the first target area. In preferred embodiments, both of the traumatizer and applicator are under the operative control of a general purpose digital computer.

Unless otherwise specified, any of the attributes, components, materials, or steps that are described with respect to one embodiment of the present disclosure (such as the disclosed methods) may be applicable to the attributes, components, materials, or steps of other embodiments of the present disclosure (including the disclosed systems).

The present system comprises at least one traumatizer for injuring a first target area at the body surface. The target area may be some part or the entirety of a physical feature or may some location relative to the physical feature. The traumatizer may include any one or more modalities that are suitable for inducing regeneration, remodeling, resurfacing, restoration, follicular neogenesis, neocollagenesis, stem cell recruitment, activation, or differentiation, reepitheliazation, wound healing, or any other desired biological or physical modification. The traumatizer may be configured to injure the target area by mechanical, chemical, energetic, sound- or ultrasound-based, or electromagnetic means.

The system is preferably configured to allow the traumatizer to be moved in any direction relative to the body surface. For example, the traumatizer may be associated with a movable element, such as an arm or other mounting or housing, that may be moved relative to the body surface under mechanized or manual (human) manipulation. The operation of the traumatizer (e.g., its activation, deactivation, and movement thereof) may be under human, machine (e.g., computer), or mixed human and machine control. The components that may be necessary for moving a device such as the traumatizer to any point on a two dimensional plane (corresponding to any point on the body surface), as well as any point in three dimensional space (and thereby any point in space relative to the body surface) are readily identified by those of ordinary skill in the art.

The traumatizer may be any device that is capable of effecting the removal of a column of tissue at the target area to form a channel. For example, the removal of a column of tissue at the target area may be accomplished by a fractional ablative laser, a punch biopsy needle, a microneedle, a micro-coring needle, or another suitable modality.

Other traumatizers may invoke a microdermabrasion model that induces reorganization of existing body surface components. Where the body surface is skin, such components may include follicular structures. The microdermabrasion model is substantially superficial and may have a clinical endpoint that is characterized by pinpoint bleeding. Where the body surface is skin, the microdermabrasion model may include removal of the stratum corneum and epidermis. Standard dermabrasion may be used to achieve the desired clinical endpoint in this injury model. Disruption of the body surface in this manner may be induced by using a device (e.g., sandpaper, a felt wheel, a supersonically accelerated mixture of saline and oxygen, tape-stripping, peels, pumice pads, Scotch-Brite pads, or microneedles). Alternatively, disruption may be induced using a chemical (e.g., phenol, trichloracetic acid, or ascorbic acid, or a protease including papain, bromelain, stratum corneum chymotryptic enzyme, trypsin, dispase, or thermolysin), ultrasound, acoustic radiation, or electromagnetic radiation (e.g., electroporation). In one aspect, disruption does not result in disturbance to the stratum corneum or upper epidermis. Lasers may be used to invoke the microdermabrasion model as well (whether the body surface is skin or another surface). Standard CO₂ or YAG/Erbium lasers may be used for this purpose by selecting the appropriate depth of body surface disruption; for skin, this involves the removal of the stratum corneum and epidermis. In other embodiments, the traumatizer may configured to propel particles against the body surface in order to effect removal or resurfacing at a target area. Configurations of this type are disclosed, for example, in U.S. Pat. Nos. 6,306,119, 6,726,693, and 6,764,493 (disclosing skin resurfacing and treatment using biocompatible materials).

Another type of traumatizer may be configured to accomplish the segmentation of a hair follicle into at least two disunited subunits. The traumatizer may include an incisor that is applied at an oblique angle relative to the body surface to a depth below the body surface that is sufficient to intersect and cross the follicle. In some embodiments, incisor is applied at an angle of 89°, 85°, about 80°, about 75°, about 70°, about 65°, about 60°, about 55°, about 50°, about 45°, about 40°, about 35°, about 30°, about 25°, about 20°, about 15°, about 10°, about 5°, or less relative to the body surface. The incisor may be any physical instrument, material, or form of energy that segments the follicle into at least two disunited subunits. For example, the incisor may be an ablative laser, a punch biopsy, a microneedle, or a micro-coring needle that results in the removal of a column of tissue to form a channel that transects the follicle. The incisor may also be a non-ablative laser that leaves a coagulum along its path but likewise transects and segments the follicle. In other embodiments, the incisor may be a high-pressure jet of fluid, such as water or gas, that penetrates the body surface and segments the follicle. The traumatizer may be configured so that the incisor may be applied at an angle φ relative to axis y that is perpendicular to the body surface, wherein the hair follicle is oriented at an angle α relative to the body surface, wherein the sum of angle α and an angle β is 90°, and wherein the sum of angle φ and an angle β is about 65° to about 115°. In some instances, the sum of angle φ and angle β may be about 70°, about 75°, about 80°, about 85°, about 90°, about 95°, about 100°, about 105°, or about 110°.

The segmentation of a hair follicle by applying an incisor at an oblique angle relative to the body surface may alternatively comprise splicing a hair follicle substantially along its long axis. For example, given a hair follicle that is oriented at about 40° relative to the body surface, the incisor may be directed at a comparable angle against the body surface at the location of the follicle and parallel to the long axis of the follicle. The application of an incisor in this manner preferably functions to splice the follicle along its long axis into at least two portions (if two portions are produced, halves). Each portion of the spliced follicle contains all of the biological follicular components that are necessary to generate a complete follicle and produce hair. Thus, the splicing of a hair follicle in this manner can generate a pair of hair-producing follicles from a single follicle.

Optionally, further to the process of segmenting a hair follicle by applying an incisor at an oblique angle relative to the body surface, an incisor may also be applied substantially “downwards”, i.e., at about 90°, relative to the body surface in order to segment a further hair follicle that is oriented at a substantially similar angle relative to the body surface. The application of an incisor substantially downwards onto a hair follicle having this orientation preferably functions to splice the follicle into at least two substantially vertically oriented halves. Each half of the spliced follicle contains all of the biological follicular components that are necessary to generate a complete follicle and produce hair. Thus, the splicing of a hair follicle in this manner can generate a pair of hair-producing follicles from a single follicle.

The present systems may further comprise an applicator for delivering a composition to the first target area. The applicator may be any appropriate device for delivering compositions of the variety disclosed herein. The applicator may be configured for contacting the body surface with a composition by spraying, dripping, painting, propelling, misting, atomizing, or injecting, or may be configured for applying the composition by any combination of such methods. The application of the composition to the target area may be topical, may be to some location at the target area that is interior to the body surface, or both, and the applicator may be configured accordingly. In some embodiments, applicator is configured to deliver a composition that is a fluid onto the target area. Nozzles for dripping, misting, atomizing, or stream-spraying (e.g., in a flat or round stream) a fluid are well known in the art. The applicator may be configured for “painting” a composition onto the body surface, for example, as a brush, roller, or roller ball. Applicators for injecting a composition at the target area include needles, such as nano- or micro-injection needles. The applicator may be configured for applying a composition by iontophoresis, ultrasound penetration enhancement, electroporation, sponge application, or by any other suitable process. Preferably, the applicator is configured so that the delivery of the composition to the location of the target area is spatially precise within a therapeutically acceptable margin of error. Exemplary devices for the propulsion of compositions comprising particles are disclosed in U.S. Pat. Nos. 6,306,119, 6,726,693, and 6,764,493, as well as WO 2009/061349.

The composition may comprise components that cause gelling or hardening of the composition (for example, the gelling or hardening may occur as a result of a reaction between two or more components within the composition), and the applicator may be configured for delivering a composition of this kind. To this end, the applicator may comprise a mixer for combining two or more gel-forming components prior to delivering the composition. The formation of the gel after the mixing of the gel-forming components may be delayed long enough for the composition to be delivered as fluid to the target area, or the gel may form substantially immediately after the mixing of the gel-forming components but either the gel may be capable of undergoing shear-thinning such that the gel may still be sprayed or otherwise delivered by the applicator, or the applicator may be configured for delivering a gel.

In other embodiments, the applicator may comprise components that substantially correspond to those used in inkjet technology. Thermal inkjets, piezoelectric inkjets, and continuous inkjets are the three main versions of this technology, and the components for the applicator may substantially correspond to those used in any of these types of inkjet systems. In such embodiments, the system may be configured to coordinate the activity of the traumatizer with that of the applicator. For example, the system may be configured to instruct the applicator to apply the composition to the precise spatial position of the site of injury that was induced by the traumatizer; where the traumatizer removes a column of tissue at the target area to form a channel, the system may be configured to instruct the applicator to apply the composition into the channel. The system may be configured in this fashion through the use of computer software that determines the spatial position of the traumatizer at the time of injury and correlates this position to the precise site of injury and the location of the resulting channel, and then positions the applicator so that the composition is precisely directed into the channel using the inkjet technology.

The present systems may further comprise components that are capable of displacing or eliminating an impediment; such components may generally be referred to as “displacers”. A hair, a sweat droplet, oil, dirt, a mole, skin pigmentation, dead skin, a scab, or any combination thereof may be located at the body surface in such a manner as to constitute an impediment to assessment, treatment, or both. In a hair restoration applications, it may be important for the skin perturbation modality leaves existing hair and hair follicles to remain intact. This may especially be the case when treating areas of thinning hair as opposed to areas of total baldness (e.g., as in the case of female diffuse alopecia). As such, when treating to restore hair, an objective is typically not to remove hair that may already be present.

In some instances, an impediment may be associated with a physical feature (e.g., in physical proximity to a physical feature) and may have the potential for interfering with assessment, treatment, or both of the physical feature. Even if the impediment does not interfere with assessment or treatment of the body surface, it may be desirable to avoid injuring the impediment. For example, if the impediment is a hair and the treatment involves the use of a laser, it may be desirable to avoid severing or otherwise damaging the hair, especially of an objective of the treatment is to promote hair growth or to increase the density of hair. A computer may be loaded with the appropriate software for activating a displacer after each step of selecting a further target area and prior to each injuring step, for activating the displacer at other regular intervals (e.g., after every other step of selecting a further target area), or for activating the displacer at random intervals during an iterative process of selecting and injuring further target areas.

Depending on the type of impediment that is more likely to be present at the body surface undergoing treatment, any of a variety of different approaches may be used to displace or eliminate the impediment. For example, forced air may be used to blow away, blow aside, or evaporate an impediment; a hair or a sweat droplet may be blown aside, dead skin or dirt may be blown away, and a sweat droplet may be evaporated. A gas jet can be readily generated via a disposable CO₂ cartridge, and embedded software via the micro-controller can gate a solenoid valve that fires the gas. An exemplary process may include (1) selection of a target area; (2) firing the burst of gas to attempt to displace any impediment that might be present at the target area; (3) firing a laser (or otherwise imposing injury) as described above. Additionally, if the traumatizer includes a biopsy needle array or micro-needle array, then gas jets could be delivered down the center lumen of the needles to displace a hair or another impediment distally as the needle enters the skin. A stream of liquid, such as water, may also be used to displace an impediment. Devices for producing forced air (e.g., in a stream), a stream of liquid, or other suitable means for displacing or eliminating an impediment may be readily appreciated among those skilled in the art.

Displacers may be separate from or integrated with any of the other components described herein with respect to the present systems. For example, the applicator or traumatizer may themselves be used to displace or eliminate an impediment; the applicator, traumatizer, or both may be equipped, for example, to deliver a stream of air or liquid. In other instances, the applicator and traumatizer are not themselves equipped to perform displacement or elimination of an impediment, but there may otherwise be a device associated with (e.g., occupying the same mounting as) either of these components that is capable of performing these tasks. In yet other embodiments, the displacer is separate from the applicator and traumatizer. Any method or device for displacing or eliminating an impediment may be used in accordance with the present disclosure.

The components of the present systems may be substantially separate or may be integrated into a unitized structure. Any subset of the system components may be integrated (e.g., a displacer and a traumatizer), or all of the components may be substantially separate.

In accordance with some embodiments of this invention, unitary or cooperative devices may be employed to achieve desired action upon such surfaces. Thus, a plurality of functions may be integrated into a single ‘head’ or into a plurality of ‘heads’ which cooperate with each other, such as under control of a computer or operator, to achieve desired actions upon the surfaces. The functions which may be integrated include, among other things, injuring, displacing, and composition applying. For some embodiments, displacing and injuring, such as with a laser and composition applying, such as by ‘ink jetting’ techniques are integrated together into a single ‘head.’ The apparatuses may be effectively miniaturized such that the working head carrying them may be introduced into the corpus of a subject through arterial access. Larger heads may be used where convenient for the intended uses.

The composition delivery orifices may deliver a large variety of liquids including water, aqueous therapeutic solutions or slurries, liquid pharmaceuticals, dyes, indicators, radioopacifiers, radiotherapeutic absorptive materials, such as for subsequent application of radiofrequency, magnetic or other energy, or other things, as provided supra and appreciated among those having ordinary skill in the art. Such liquids may include liposomes, polymersomes, nanoparticles or other things for the delivery of drugs or therapeutic agents to specific locales. In one embodiment, one or more dispensing orifices are disposed to as to rinse or clean the injuring, e.g., laser, portion of the head, or other things so as to provide an unimpeded field of action for the devices comprising the head.

An example of one integrated head is shown in FIG. 2. An integrated head 10 comprises a body 14, which may be conveniently molded to include locations for placement of apparatuses for accomplishing the desired actions. Thus, a displacer 14 is included together with a surface injuring apparatus, such as a, preferred, laser 16. These may also be integrated in the head 12 with one or more fluid composition deliver orifices 18, such as “ink jets.” Control, power, sensing, fluid providing and other feeds are also provided to the internal area of the head 20, including, for example, fluid supplies 22, power supplies 24 and control circuitry 26. Several of each of these may be included as needed to effect control, powering, and materials supply to the head.

In other embodiments, a plurality of integrated (or non-integrated, but cooperative) heads may be provided to accomplish action upon surfaces. In other embodiments, one or more heads are operated under computer or robotic control. Placement apparatus, such as an X-Y positioner, many examples of which are known per se, may be used to move the head under operational control, to specific locations. Such positioners may be controlled manually by an operator, or the same may be controlled by a computer or robotic controller. Each of these is also known per se and such control is well within the skill of routineers in the art. It is particularly preferred, when employing a positioner for the head or heads, to provide common control between the head and the positioner to enable action at a selected surface location to cooperate with positioning of the head at that location. Serial positioning and action accomplishment may be attained thereby and will accord convenience and efficacious action.

As one example of the use of an integrated head in accordance with one embodiment of this invention, cardiac ablation may be accomplished without thoracic surgery. A miniaturized head integrating displacer, laser lens, and composition delivery orifice is introduced into the femoral artery of a patient in need of cardiac ablation. The head is connected with a flexible ‘tail’ through which run fluidic, power and control tubes and circuits. Such are interfaced with a control unit to effect control of the functions performed at the head. The head is moved into the body of the heart under control of a surgeon, such as by employing fluoroscopy, in order to position the head at the location where ablation is needed. A positioner on the integrated head may confirms location, whereupon the injuring device, in this case, a laser, is activated to remove material from the selected location of the heart. A therapeutic, anaesthetic, palliative, anti-infective, protective or other material may be applied to the injured location via the delivery orifices. The head may then be moved under guidance from a positioner and further laser ablation performed until the surgeon is satisfied with the extent of the treatment. This procedure may be repeated over time since access, via the artery, is relatively benign compared with thoracic surgery.

A somewhat larger head may be used for colonic access since access through the rectum may be done similarly to colonoscopy. The colon is cleansed in a conventional way and the head inserted into the colon. Imaging of the interior of the colon may be done via a camera and features of interest, such as polyps, identified. The polyps may be ablated with a laser or other injuring device. In the case of the laser, cautery accompanies the ablation. Large pieces of the polyp may simply be left behind in the colon for evacuation later. In may be desired to provide a further device to cooperate with the head. Thus, a gripper may be included with the head or separately introduced to retrieve samples for analysis. Fluid may be dispensed to clean the head, treat the area of the polyp or for other purposes. In this example, fluid dispensing may not be needed and the head may omit such orifices or may not actuate them.

Where any of the traumatizer, applicator, and displacer are under the operative control of a general purpose digital computer, the computer may be configured to enable the components thereof to operate in a substantially coordinated fashion, such that the operation of each of the respective components, any act of displacing or elimination by the displacer; any injuring by the traumatizer; and any selection of a further target area on the body surface having a preselected geometry with respect to the first target area, are appropriately coordinated. For example, the computer may control such aspects as the activation and deactivation of the components relative to one another; the determination of the type of composition (if any) that is applied, based on the injury that is induced by the traumatizer; or other actions that require coordination between or among components. The system may be configured to enable any pair or all of the components thereof to operate in a substantially coordinated fashion. In certain embodiments, the traumatizer and applicator are operatively linked via general purpose digital computer.

Where any of the traumatizer, applicator, and displacer are under the operative control of a general purpose digital computer, software may be used to instruct any or all of the traumatizer, applicator, and displacer to function in accordance with a protocol that is adapted for use in connection with a particular type of body surface and/or known characteristics of a particular body surface. For example, if the body surface is the scalp, the software may include a protocol that instructs the traumatizer to induce the types of injury that are most suitable for achieving a purpose that is needed for the scalp, e.g., to invoke a microdermabrasion model to foster follicular neogenesis. Likewise, if the body surface is a scalp, the protocol may include instructions for the applicator that permit coordination with the traumatizer, such as by instructing the applicator to deliver a composition in a manner and of the type that is appropriate for the type of injury that was formed by the traumatizer. The system may also be configured to allow a practitioner to input various characteristics about the body surface that is to be subjected to treatment in order to further refine the protocol that is implemented with respect to the body surface. For example, where the body surface is a scalp, a practitioner may visually assess the scalp to determine certain characteristics thereof, such as the terminal hair density, the absence or presence and frequency of certain physical features such as age spots, scars, sweat glands, and the like, and/or of certain types of impediments, and the practitioner may subsequently input such information into the computer. The computer may then use such information to select a protocol that takes into account the absence or presence and frequency of the physical features and/or impediments and instructs one or any combination of the traumatizer, applicator, and displacer accordingly. For example, if the practitioner's assessment of a scalp reveals a very low density of terminal hairs and a high frequency of age spots, then the practitioner can provide such information to the computer, which will then select a protocol that instructs the traumatizer to induce injury and optionally apply composition that is consistent with treatment for promoting follicular neogenesis, and to induce injury and optionally apply composition that is consistent with treatment for reducing the appearance of age spots.

The disclosed methods and systems therefore allow for the iterative induction of therapeutic injury to a body surface in order to provide an unprecedented degree of treatment efficiency that is distinguishable from that of prior methodologies, which do not provide systems that allow for such processes on a methodical basis.

### Example 1

### Method of Treatment of Skin

A method according to the present invention for effecting treatment of the skin on a human scalp is performed as follows. A subject with near complete hair loss and mild dyspigmentation on the scalp is seated in a stationary examination chair.

Next, using a programmed protocol for treating physical features that are known to be present on the subject's scalp (the practitioners having assessed the subject's scalp, determined that germane features included “featureless” areas of skin, age spots, terminal hairs, and sweat glands, and provided such information to the computer in order to trigger the use of an appropriate protocol), the computer positions a fractional laser above the scalp, and the laser is activated for a prescribed time and at a prescribed power for removal of a column of tissue at the target area to a depth of about 1 mm, thereby forming a channel at the location of injury. Using a protocol that is appropriate for an area in which a channel has been formed, the computer positions an applicator at a location above the scalp that corresponds to the site of injury in the second further target area. The applicator includes an inkjet-type head for delivering a composition substantially directly into the channel. A small volume (about 50 μL) of a composition comprising 6-bromo-indirubin-3′-oxime (a GSK3β modulator) and carrier comprising PEO-PPO-PEO (a thermoreversible polymer that gels when exposed to human physiological temperatures) is delivered as a fluid to the location.

The computer then uses the preprogrammed protocol to select a new target area on the portion of the scalp that is precisely 4 mm “above” (i.e., at a 90° angle from) the first target area. The selection of the further target area is in accordance with a preset directive that instructs the computer to select further target areas from a rectilinear grid defined by points that are separated from one another by 4 mm.

Next, using the programmed protocol for treating a scalp having the characteristics described above, the computer positions an applicator that is configured for propelling particles at a location above the scalp that corresponds to the further target area, and the applicator is activated for a prescribed time to deliver lithium-containing particles at a prescribed velocity (calculated to penetrate the skin to a depth of 1 to 3 mm) at the location of the further target area. Using a protocol that is appropriate for an area that has been bombarded with lithium-containing particles, the computer positions a second applicator at a location above the scalp that corresponds to the further target area. The applicator includes a spray nozzle for delivering a composition to the body surface. A small volume (about 50 μL) of a composition comprising aminoxidil and an appropriate excipient is delivered as a fluid to the location.

The computer again uses the preprogrammed protocol to select a new target area (a “second further target area”) on the portion of the scalp that is precisely 4 mm “above” (i.e., at a 90° angle from) the further target area. In accordance with the preprogrammed protocol for treating a scalp having the characteristics described above, the computer again positions a fractional laser above the scalp, and the laser is activated for a prescribed time and at a prescribed power for removal of a column of tissue at the second further target area to a depth of about 1 mm, thereby forming a channel at the location of the second further target area. Using the protocol that is appropriate for an area in which a channel has been formed, the computer positions an applicator at a location above the scalp that corresponds to the site of injury in the second further target area. The applicator includes an inkjet-type head for delivering a composition substantially directly into the channel. A small volume (about 50 μL) of a composition comprising 6-bromo-indirubin-3′-oxime and carrier comprising acrylate-lactate-PEO-PPO-PEO-lactate-acrylate is delivered as a fluid to the location.

The computer again uses the preprogrammed protocol to select a new target area (a “third further target area”) on the portion of the scalp that is precisely 4 mm “above” (i.e., at a 90° angle from) the second further target area. Once again using the programmed protocol for treating a scalp having the characteristics described above, the computer positions the applicator that is configured for propelling particles at a location above the scalp that corresponds to the third further target area, and the applicator is activated for a prescribed time to deliver lithium-containing particles at a prescribed velocity (calculated to penetrate the skin to a depth of 1 to 3 mm) at the location of the further target area. Using a protocol that is appropriate for an area that has been bombarded with lithium-containing particles, the computer positions a second applicator at a location above the scalp that corresponds to the third further target area. The applicator includes a spray nozzle for delivering a composition to the body surface. A small volume (about 50 μL) of a composition comprising aminoxidil and an appropriate excipient is delivered as a fluid to the location.

The described process is performed iteratively to give rise to additional target areas, wherein the additional target areas form a rectilinear grid relative to the portion of scalp.

What is claimed:

1. A method for treating a preselected body surface comprising:
(a) injuring a first target area on the body surface;
(b) optionally applying a composition to the injured first target area;
(c) selecting a further target area on the body surface having a preselected geometry with respect to the first target area;
(d) injuring the further target area on the body surface; and
(e) optionally applying the same or a different composition to the injured further target area.

2. The method of claim 1 wherein steps (c)-(e) are performed iteratively to give rise to one or more additional target areas.

3. The method of claim 2 wherein the target areas form a pattern upon or relative to said body surface.

4. The method according to claim 1 wherein said injury comprises segmenting a hair follicle at said first target area, said further target area, or both into at least two disunited subunits.

5. The method according to claim 4 wherein said first target area, said further target area, or both are injured by applying a first incisor to said target area at an oblique angle relative to said body surface.

6. The method according to claim 5 wherein said first incisor is a laser.

7. The method of claim 1 wherein said body surface is skin.

8. The method according to claim 1 wherein said body surface is an internal body surface.

9. The method of claim 1 wherein said first target area, further target area, or both are injured by removing a column of tissue at said target area to form a channel.

10. The method of claim 9 wherein said channel extends from the body surface to a depth of about 0.5 mm to about 4 mm below said surface.

11. The method according to claim 1 wherein said composition comprises a fluid.

12. The method according to claim 11 wherein said fluid comprises a dye.

13. The method according to claim 1 wherein said composition forms a gel following application of said composition to a target area.

14. The method according to claim 13 wherein said gel releases an active ingredient over time.

15. A system for treating a body surface comprising:
a traumatizer for inducing injury to a first target area at said body surface;
an applicator for delivering a composition to said first target area;
wherein said traumatizer, said applicator, or both are under the operative control of a general purpose digital computer; and
wherein said computer is configured for selecting a further target area on the body surface having a preselected geometry with respect to the first target area.

16. The system according to claim 15 wherein said applicator is configured for delivering a fluid.

17. The system according to claim 16 wherein said applicator further comprises a mixer for combining two or more gel-forming components and a physiologically active ingredient prior to delivering said composition.

18. The system according to claim 15 wherein said applicator comprises components that substantially correspond to those used in inkjet technology.

19. The system according to claim 15 wherein said traumatizer is configured for applying a first incisor at an oblique angle relative to said body surface.

20. The system according to claim 19 wherein said first incisor is a laser.

21. The system according to claim 15 wherein said traumatizer comprises a microneedle or a micro-coring needle.

22. The system according to claim 15 wherein said computer is configured for controlling the activation of said applicator relative to the activation of said traumatizer.

23. The system according to claim 15 wherein said computer is configured for performing said selection iteratively to give rise to one or more additional target areas.

24. The system according to claim 15 wherein traumatizer and applicator are integrated into a unitized structure.

25. The system according to claim 15 wherein the traumatizer and applicator are operatively linked via general purpose digital computer.
